SEQUENCE LISTING (13 sequences)

SEQ ID NO: 1 - DNA, 2885 bp, Homo sapiens
ttttttttttttttttttaatagttgacactcaattttattgctaaaaaaaatgtcctctggagtgacagcaggtatttgtcctcagcctctccccagagtccctgggagtgtttctgttattgtggagggaataggggatctggggacagggtgggacgtaccatggggacaggaggtcacagaacacacacacacacacacacacacacacacacacacgtgcacacacacatccccccacaactacagccgccaaaataaccagcatggtgttgtaacatccccccagtgggggctagaattccccatggtgacctgtgacctgctccctgagacaggggaggccaggcaggtcacggtggggacgcagcagaacattatttctccatcttgctgttttctagccttgagttggggacatgagtgagaactgggggttttgaggagtaacccttgtgaacacatgtactccagcagccaggcatcccagatctcctgtcctggagggtgctggggcccctggctccccagagtgtgcaggcagacccccagagccctagctcatccatttatccattcctcataatccagtgtccaaagagcacccccagcagggcagggaaggtccctcccggggtttacatgactgattccttctcagaggcgaccgtggcatcccctgcgggcccccgatagtgtttgaggagggggtttccttcctcagtggggactggcagcggatccaggggcagctcactcttcacttgtatcaactcaggctctgtgcttctcgactccgtacggtccacataattttggaggagtcctgcccccagagcgtcaccttctacattgaggacggtacaggaccggtcgactagccagtccacagccaggatcaaggagatatggtcgaccgggaggttgactgcttcgaggatgatggccagagtgaggacacctccagcagggatgcccgctgcccccacgctggacgctgtggccgtgaccaggatggtgatgatctttacgaagtccaaggactgctggctgagctgtgcaatgaacactgcggccacgcactggaagagcgcggcaccgtccatgttgacggtggcgccgatgggcaggatgaaacggctgatgtgcttggccacgccattattctcctccacgcacttcatcatcagcggcagcgtggcggaactggaagaggtcccaaaggcagtggccagcggcgtcacgatgccccacaggaagcggtaggggtttttgcgggtgaagaggaagtagatgaggggcagtaccaggagcccatggatggcgtgacccagcaggcagcacagaatgtacttgccaaggcgggcaaagagtaaacccacatcctccatctccacgatcttgccagccaccaggaacatgatgcccacaggggcgtaccacatgatccaggagaccagaaccatggtggcctcattgaaggagttgaagaagcggataagcagctccccttcaggccccagcttccgcagcgccacaccaaagacgatggcaaacactaccaagcccaggatgttcatcccctccacctcctgccccacgggcaccttcaccctggttccggtgatattcctctcttcataggtggtagagtatgagcgaaaggctgctgacaccaggttggaagggaagatatttctcgcaagatccaggaacgaatcgagcacctccttgctgggggcattttcggcactgcccgcggctcccacggaggcgttgatggcggcggaggcggcgcccggctgcagagccagcgccaagcccactccgagcgccgacgccagcagcgtggtgaccaggaaaaagagcagcgcccaggcgcccagacggccgagcgcgccggggtccaggctggcggcgccgccgatcaagctgcacaccaccagcggcaagatgatcatccgcagcagacgcagcagcagctcgcccgggaagacgaaggcctcaagcgctcccgggcccaacgccagcgcacccccggcccccgacacccccagtcccagcgccacgccggccaccacggccaccactgtcagcagcacaagcaggttggctcgaaggcagcggcgcaccaggtcccgggaaccgcagtagccgcctgctgccgcgccttggtcctcgatggaggccagctgccaggcccccgttggcggtggctccgccgctgcgagccccttggagtctcgaggaggatcggccaccatgatgggaagcaccggggtttcttagcgcctggaagctggctgggagcgcttgggctccttcccaggacccgacgttcctaggactgagttgagtaacagcacctggagactggaactttggagggctccttagagttgtgagttcacagcactgaagttccttggctcttggaagctggagtgtttaaattccccaggctgggcgctgaggcttctctgctctgccccgtgtgccagatgtccgaaagctgggagttcggagcgcccgggttccttggccctaggagctgggaatacgagagtttctctgggtgggacgtggggcccttggctccaggaggttgagtgcccccagatggcggaggtctgcaggaggctaggttctggtggcgcagatcccggacgcctggatccgggaggcggggacccgggctgcctgggtcttggacactgagggctgggatgccagaatctgggggccgggaagcggactccggaaacaggggacccaggctcttaggtccgggagggtgggatgcgggcccctgtgtacggaaggcggtggtctggtgtccgggagtagcggttaccagccagagaaagcctcccgggcgtgccg

SEQ ID NO: 2 - DNA, 1333 bp, Homo sapiens
ctgcgggcccccgatagtgtttgaggagggggtttccttcctcagtggggactggcagcggatccaggggcagctcactcttcacttgtatcaactcaggctctgtgcttctcgactccgtacggtccacataattttggaggagtcctgcccccagagcgtcaccttctacattgaggacggtacaggaccggtcgactagccagtccacagccaggatcaaggagatatggtcgaccgggaggttgactgcttcgaggatgatggccagagtgaggacacctccagcagggatgcccgctgcccccacgctggacgctgtggccgtgaccaggatggtgatgatctttacgaagtccaaggactgctggctgagctgtgcaatgaacactgcggccacgcactggaagagcgcggcaccgtccatgttgacggtggcgccgatgggcaggatgaaacggctgatgtgcttggccacgccattattctcctccacgcacttcatcatcagcggcagcgtggcggaactggaagaggtcccaaaggcagtggccagcggcgtcacgatgccccacaggaagcggtaggggtttttgcgggtgaagaggaagtagatgaggggcagtaccaggagcccatggatggcgtgacccagcaggcagcacagaatgtacttgccaaggcgggcaaagagtaaacccacatcctccatctccacgatcttgccagccaccaggaacatgatgcccacaggggcgtaccacatgatccaggagaccagaaccatggtggcctcattgaaggagttgaagaagcggataagcagctccccttcaggccccagcttccgcagcgccacaccaaagacgatggcaaacactaccaagcccaggatgttcatcccctccacctcctgccccacgggcaccttcaccctggttccggtgatattcctctcttcataggtggtagagtatgagcgaaaggctgctgacaccaggttggaagggaagatatttctcgcaagatccaggaacgaatcgagcacctccttgctgggggcattttcggcactgcccgcggctcccacggaggcgttgatggcggcggaggcggcgcccggctgcagagccagcgccaagcccactccgagcgccgacgccagcagcgtggtgaccaggaaaaagagcagcgcccaggcgcccagacggccgagcgcgccggggtccaggctggcggcgccgccgatcaagctgcacaccaccagcggcaagatgatcatccgcagcagacgcagcagcagctcgcccgggaagacgaaggcctcaagcgc

SEQ ID NO: 3 - DNA, 19 bp, Homo sapiens
aggaggtgctcgattcgtt

SEQ ID NO: 4 - DNA, 19 bp, Homo sapiens
agcacatcagccgtttcat

SEQ ID NO: 5 - DNA, 19 bp, Homo sapiens
tcctcgaagcagtcaacct

SEQ ID NO: 6 - DNA, 19 bp, Homo sapiens
gagcctgagttgatacaag

SEQ ID NO: 7 - DNA, 20 bp, Homo sapiens
aatttaggtgacactataga

SEQ ID NO: 8 - DNA, 20 bp, Homo sapiens
ggaactgcaggatccaaaga

SEQ ID NO: 9 - DNA, 20 bp, Homo sapiens
aggtcagcaggaggtggatg

SEQ ID NO: 10 - DNA, 20 bp, Homo sapiens
atggatcccatggaactgag

SEQ ID NO: 11 - DNA, 20 bp, Homo sapiens
ggccatggcataggacaagc

SEQ ID NO: 12 - DNA, 18 bp, Homo sapiens
ccgctgatgatgaagtcg

SEQ ID NO: 13 - DNA, 18 bp, Homo sapiens
cccccgatagtgtttgag

(19) United States
(12) Patent Application Publication
(10) Pub. No.: US 2006/0280725 A1
(43) Pub. Date: Dec. 14, 2006

(54) Compositions and methods of treating and diagnosing hepatoma

(76) Inventor: Barrie Bode, Chesterfield, MO (US)

Correspondence Address: Joseph E Zahner, 3556 Caroline mall, Suite 208, St. Louis, MO, 63104, US

(21) Appl. No.: 10/563,339

(22) PCT Filed: Jun. 30, 2004
(86) PCT No.: PCT/US04/21101

§ 371 Date: Jan. 3, 2006

(30) Foreign Application Priority Data
Jul. 2, 2003 (US) 60484728

(51) Int. Cl. A61K 48/00; A61K 39/395; C12N 15/861

(52) U.S. Cl. 424/93.2; 424/155.1; 514/44; 435/456

(57) ABSTRACT

Disclosed are pharmaceutical compositions and methods of treating hepatoma by administering to patients or other populations of cells agents that selectively inhibit the uptake of glutamine by hepatocarcinoma cells, causing the concomitant apoptosis of hepatocarcinoma cells. Preferred agents target the amino acid transporter B0 (ATB0) protein for inhibition. Disclosed are antisense polynucleotides that reduce the expression of ATB0 in hepatocarcinoma cells, causing those cells to reduce the uptake of glutamine and subsequently undergo selective apoptosis.

### GOVERNMENT SUPPORT

This work was supported in part by a grant from the National Institutes of Health (Grant No. CA69505). The United States Government has certain rights in this invention.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

This invention relates generally to compositions useful for the selective killing of carcinoma cells, and specifically to compositions useful for the selective killing of hepatocarcinoma cells through the selective inhibition of a ATB⁰ gene product.

2. Description of the Related Art

According to the American Liver Foundation, hepatocellular carcinoma (“HCC”) is the most common primary malignant tumor of the liver. HCC is also the leading cause of cancer death in Africa and the developing world (Ogunbiyi, J. O., Seminars in Oncology 28(2):179-182, 2001)) and its prevalence in Europe and the United States is on the rise due to increased incidence of viral hepatitis. Currently there is no effective treatment for HCC other than resection or transplant, and both modalities are often unsuccessful.

In HCC, there is a net increase in glutamine consumption (Bode and Souba, J. Parent. Enteral. Nutr. 23:S33-37, 1999), which subverts the systemic glutamine homeostatic function of the liver and results in significantly decreased plasma glutamine levels in patients with liver cancer (Hirayama, et al., Biochem. Med. Metab. Biol. 38:127-133, 1987)—an event that can adversely impact glutamine-dependent cells such as those in the immune system (Abcouwer, S. F., Nutrition 16:67-69, 2000). It has been recognized that the transport of glutamine across the plasma membrane of liver cells may represent a rate-limiting step in liver cell metabolism, especially when intracellular catabolism is accelerated (Haussinger at al., Eur. J. Biochem. 152:597-603, 1985; Low et al., Biochem. J. 295:617-624, 1993). In normal rat and human hepatocytes, glutamine transport is predominantly mediated by a Na⁺-dependent transporter with narrow substrate specificity (glutamine, histidine and asparagine) termed System N (Bode et al., Hepatology 21:511-520, 1995; Kilberg, Handlogten, Christensen, J. Biol. Chem. 255:4011-4019, 1980). Two separate genes encoding for this activity, termed SN1 and SN2, have recently been isolated (Fei et al., J. Biol. Chem. 275:23707-23717:2000; Nakanishi et al., Biochem. Biophys. Res. Comm. 281:1343-1348, 2001). Previous work of applicant's laboratory showed that human hepatoma cells take up glutamine at rates 10- to 30-fold faster than normal human hepatocytes via a transporter historically termed System ASC (Bode et al., Hepatology 21:511-520, 1995; Bode and Souba, Ann. Surg. 220:411-424, 1994; Bode and Fuchs, Gastr. Liver Phys. 283(5):G1062-1073, 2002). This high affinity Na⁺-dependent glutamine transport activity is not expressed in normal hepatocytes (Bode et al., Hepatology 21:511-520, 1995).

Additional studies have demonstrated that glutamine is important for hepatoma cell survival. For example, it has been shown that glutamine synthetase rates are depressed and glutamine oxidation and glutaminase rates are increased in human hepatomas compared to normal livers (9Bode and Souba, 1994; Matsuno, T., J. Cell. Physiol. 148:290-294, 1991; Matsuno and Goto, Cancer Res. 52:1192-1194, 1992; Bode and Fuchs, 2002). This observation that many tumor cells exhibit enhanced rates of glutamine metabolism has led to the testing of glutamine analogs as anti-cancer agents (Ahluwalia et al., Pharmacol. Ther. 46:243-271, 1990).

A cDNA was subsequently isolated that encodes for a broad specificity Na⁺-dependent glutamine transporter, termed ATB⁰ (Keduda et al., J. Biol. Chem. 271:18657-18661, 1996; Keduda et al, Am. J. Physiol. Gastrointest. Liver Physiol. 272:G1463-1472, 1997) whose properties match those previously reported for the System ASC-like hepatoma activity. Recent studies suggested that this System ASC activity may govern glutamine-dependent growth in the SK-Hep human hepatoma cell line (Bode et al., Surgery 124:260-268, 1998).

Based on these results, the hypothesis evolved that System ASC expression may be an integral event in human hepatocellular transformation. However, no studies have been conducted heretofore to determine the ubiquity of System ASC-mediated glutamine transport in proliferating liver cells, to identify the gene responsible for this activity, or to determine its utility in hepatocellular growth.

### SUMMARY OF THE INVENTION

The inventors have made the surprising discovery that the majority of glutamine uptake by hepatoma cells is mediated by System ASC, while “normal” hepatocytes mostly utilize System N for glutamine uptake. The inventors have also discovered that amino acid transporter B⁰ (“ATB⁰”), whose functional characteristics match those described for ASC-mediated glutamine uptake (6,12,30) is responsible for the high rates of glutamine transport observed in hepatoma cells. The inventors have also discovered that the specific down-regulation of ATB⁰ activity in a hepatoma cell culminates in the killing of the hepatoma cell by apoptosis, leading to a significant reduction in the number of hepatoma cells.

The invention provides for a method of inducing apoptosis of a cell, preferably a carcinoma cell, more preferably a hepatoma cell, by reducing the uptake of glutamine by the cell. In a preferred embodiment of the invention, the cell is contacted with agent that reduces glutamine uptake through the inhibition of ATB⁰ activity. Agents useful for the inhibition of ATB⁰ activity include antibodies specific for ATB⁰, ATB0 inhibitory molecules such as amino acid analogs that block the uptake of glutamine through the ATB⁰, antisense polynucleotides that block production of active ATB⁰, ribozymes that specifically destroy ATB⁰ RNAs, and small interfering RNAs (“siRNA”) that target the ATB⁰ RNA for destruction. Antisense polynucleotides, ribozymes, siRNAs or any other agent that comprises a polynucleotide may be encoded in a DNA sequence within a vector, which facilitates the transfer of the polynucleotide into the cell and the subsequent expression of the encoded polynucleotide in the cell. Vectors may be viral vectors, plasmids or linear polynucleotides. A preferred vector is an adenovirus vector. The cell may be ex vivo or in a patient.

Accordingly, in another embodiment the invention provides for a method of treating a hepatoma or other liver cancer in a patient, by administering an agent that inhibits glutamine uptake by hepatoma cells. Preferably, the agent acts by inhibiting the activity of a glutamine transporter protein ATB⁰ in a hepatoma cell. Preferred agents include antibodies specific for ATB⁰, ATB⁰ inhibitory molecules such as amino acid analogs that block the uptake of glutamine through the ATB⁰, and polynucleotides such as antisense polynucleotides that block production of active ATB⁰, ribozymes that specifically destroy ATB⁰ RNAs and small interfering RNAs (“siRNA”) that target the ATB⁰ RNA for destruction (e.g., SEQ ID NO:3, 4, 5 and 6), and vectors comprising DNA encoding same. A more preferable agent is a siRNA comprising a sequence as set forth in SEQ ID NO:3. A more preferable vector is an adenovirus vector.

In yet another embodiment, the invention is directed to a diagnostic kit and a diagnostic method for diagnosing liver cancer in a patient. A patient may be any vertebrate, preferably a mammal, more preferably a human. The method comprises determining the amount of ATB⁰ in the serum or liver biopse of the patient. Alternatively, the method comprises determining the amount of glutamine in the serum of the patient. The risk of having liver cancer can be inferred from the relative amounts of ATB⁰ (or fragment thereof) or glutamine, or both, in the patient.

### BRIEF DESCRIPTION OF THE DRAWINGS

FIG. 1. Amino acid inhibition analysis of glutamine transport in the Hep3B, PLC/PRF/5 and Focus human hepatoma cell lines and the THLE-5B SV40-immortalized human liver epithelial cell line. The Na⁺-dependent transport of 50 μM L-glutamine was measured as described in the Methods section in the absence or presence of the indicated unlabeled amino acids at 5 mM. Testing of cysteine (CYSH) required the presence of 5 mM dithiothreitol (DTT) as a reducing agent, so DTT was tested alone as well. Arrows indicate the relative transport rates for 50 μM L-glutamine in primary human adult (solid arrow) and fetal (open arrow) hepatocytes. The results represent the average±SD of four separate determinations (*p<0.010 versus control). The Eadie-Hofstee plots of the kinetic analyses are shown as an inset in each figure, with the dashed lines representing the resolved high and low affinity components of the composite data (solid line). The derived values of the kinetic constants for each component are given in the Results section.

FIG. 2. Northern blot analysis of ATB⁰ gene expression in human hepatoma and liver cells. Northern blot analysis was performed as described in the Methods section using α-³²P-dCTP random primer-generated probes (Megaprimie™, Amersham Corp).

Probes were generated from the human ATB⁰, albumin, alpha-fetoprotein (AFP) and insulin-like growth factor-two (IGF-II) cDNA's. The ethidium bromide (EtBr) stained gel is also shown to demonstrate relative RNA loading. Blot exposure times were as follows: ATB⁰, 48 h; albumin, 2 h; AFP, 18 h; IGF-II, 24 h.

FIG. 3. RT-PCR and restriction enzyme analysis of ATB⁰ expression in cell lines. The RT-PCR reaction was carried out as described in the Methods section. The RT-PCR product and SalI digestion of the product are shown. Analysis was performed on 1 μg of RNA from the six human hepatoma cell lines: HepG2 (HG2), SK-Hep (SKHI), THLE-SB (5B), Huh-7 (H7), PLC/PRF/5 (P5), Hep3B (H3B), Focus and the choriocarcinoma line JAR-1 from which the ATB⁰ cDNA was originally isolated. No product was obtained in the absence of reverse transcriptase in any sample (not shown). The sizes of selected molecular weight markers (mw) are indicated.

FIG. 4. Northern blot analysis of ATB⁰ expression in liver tumor and fetal tissue. A) A commercially available liver tumor blot (Northern Territory™, Invitrogen) was probed with a fall length ATB⁰ antisense transcript labeled with a nonisotopic detection system (Brightstar™ psoralen-biotin kit, Ambion) according to the manufacturer's specifications. Each hepatocellular carcinoma (HCC) sample is adjacent to a sample of uninvolved liver tissue from the same patient. B) Comparison of ATB⁰ expression in a clinical HCC biopsy and SK-Hep cells. A full-length ATB⁰ radiolabeled riboprobe was utilized for detection as described in the Methods section. Two different exposure times are shown to convey the relative ATB⁰ mRNA levels in each sample. C) Northern analysis of ATB⁰ and albumin expression in human hepatocytes, a primary hepatoblastoma and the hepatoblastoma cell line HepG2. Radiolabeled cDNA probes were generated with the Megaprime™ kit as described in the Methods section. Exposure times were: albumin, 2 h and ATB⁰, 48 h. D) Northern analysis of ATB⁰ and albumin expression in a fetal human liver RNA. The fetal age in weeks is indicated above each liver RNA sample. Radiolabeled cDNA probes were generated with the Megaprime™ kit as described in the Methods section. As no ATB⁰ mRNA was detected, the blot was probed with albumin as a positive control. The exposure times were: albumin, 24 h; ATB⁰, 96 h. All blots were washed under high stringency conditions (0.1×SSPE+0.5% SDS at hybridization temperatures). Where indicated, EtBr=ethidium bromide-stained RNA gels of the corresponding blot.

FIG. 5. Functional and molecular assessment of ATB⁰ expression in human fetal liver fibroblasts (HFLF) and cirrhotic liver tissue. A) Amino acid inhibition analysis of Na⁺-dependent glutamine uptake in human fetal liver fibroblasts. Fibroblasts were isolated and cultured as described in the Methods section, and amino acid inhibition analysis of 50 μM L-glutamine uptake was carried out as described in FIG. 1. *p<0.010 vs. control. The Eadie-Hofstee plot of the kinetic analysis is shown as an inset, with the dashed lines representing the resolved high and low affinity components of the composite data (solid line). The derived values of the kinetic constants for each component are given in the Results section. B) Northern blot analysis of ATB⁰ expression in HFLF. Radiolabeled cDNA probes were generated with the Megaprime™ kit as described in the Methods section, and hybridized to 20 μg of total RNA from HFLF. Exposure time was 24 h. C) RT-PCR and restriction enzyme analysis of ATB⁰ expression in HFLF, normal and cirrhotic liver. The analysis was carried out exactly as described in FIG. 3. Omission of RT is shown for the normal liver sample. D) Expression of ATB⁰ in biopsies from a patient with liver cirrhosis and hepatocellular carcinoma (HCC). Northern blot analysis of ATB⁰ & SN1 expression were performed on RNA extracted from the snap-frozen biopsies. RNA isolation and northern blot analysis was carried out as described in the Methods section, with full-length riboprobes used for detection of transporter mRNA. Exposure times for the blots are shown to illustrate relative expression levels for ATB⁰ in each of the samples.

FIG. 6. Effects of ATB⁰ competitive substrate inhibition on glutamine-dependent growth in human hepatoma cell lines. Each of the hepatoma cell lines was grown in DMEM+10% dialyzed FBS supplemented with 0 (circles), 0.5 (squares) or 2.0 mM (triangles) L-glutamine in the absence (open symbols) or presence (filled symbols) of a collective 20 mM excess of ASC/B0 substrates alanine, serine and threonine. Growth was measured over the next several days by the MTT assay as described in the Methods section, and are presented as the average±SD of four separate determinations. Where not shown, the error bar lies within the symbol. The glutamine requirements of each line for half-maximal growth ([GLN]₅₀) are also shown.

FIG. 7. Northern analysis of potential ATB⁰ compensatory pathways in hepatoma cell lines. Full-length radiolabeled riboprobes were generated and utilized for the transporters ATB⁰, SN1 and ATA2 while RT-PCR generated cDNA's of ˜300 bp were utilized to generate riboprobes for SN2 and ATA3 as described in the Methods section. Radiolabeled cDNA probes were generated with the Megaprime™ kit for glutamine synthetase (GS), as described in the Methods section. Blot exposure times were as follows: ATB⁰, 6 h; SN1, 6 h; SN2, 96 h; ATA2, 4 h; ATA3, 76 h; GS, 60 h. Cell lines that were resistant to ATB⁰-targeted growth inhibition (FIG. 6) are indicated with an (R). The relative densitometric volumes of the corresponding mRNA band intensities for each cell line are shown below the northern exposures, and were generated using the Kodak EDAS 290 system with 1D-Image Analysis Software. In all cases, the highest intensity band was set to a value of 1.0, with all subsequent intensities expressed as the percentage of the highest value. In analysis of transporter expression marked with an asterisk (*), normal human liver RNA was used as a positive control rather than isolated hepatocyte RNA.

FIG. 8. Northern blot analysis of antisense ATB0/ASCT2 expression. Total RNA was isolated as described in the methods section from cultures of the SK-Hep parent cell line and SK-Hep cells stably transfected with a 1.3 kb ATB⁰ construct in the sense orientation (Sense 2-1) or antisense orientation (Antisense 1-1) after treatment with inducer (mifepristone (MFP), 10 nM) or vehicle (ethanol, 0.1%) for the indicated times. After size fractionation by denaturing agarose gel electrophoresis (20 μg RNA/lane) and transfer to a nylon membrane, the blots were hybridized to a full length 2.9 kb sense ATB⁰ radiolabeled RNA probe generated by in vitro transcription with T7 RNA polymerase. A second hybridization with a radiolabeled 334 bp β-actin RNA probe is shown along with the ethidium bromide stained agarose gels to indicate relative loading of the RNA. Exposure times were 96 h for the ATB0/ASCT2 antisense RNA signal and 5 h for the β-actin mRNA signal.

FIG. 9. Northern blot analysis of endogenous ATB0/ASCT2 mRNA expression. Total RNA was isolated from cultures of SK-Hep cells stably transfected with a 1.3 kb ATB⁰ construct in the sense orientation (Sense 2-1) or antisense orientation (Antisense 1-1) after treatment with inducer (NFP, 10 nM) or vehicle (ethanol, 0.1%) for the indicated times. After size fractionation by denaturing agarose gel electrophoresis (20 μg RNA/lane) and transfer to a nylon membrane, the blots were hybridized to a fall length 2.9 kb antisense ATB⁰ radiolabeled RNA probe and a radiolabeled 334 bp β-actin RNA probe both generated by in vitro transcription with SP6 RNA polymerase. Exposure times were 4 h for the ATB⁰ mRNA signal and 5 h for the β-actin mRNA signal. Autoradiographs were quantified using the Kodak EDAS 290 system with one-dimensional image analysis software. ATB⁰ band intensities were normalized to those of β-actin, with the time zero ratio set at a value of 100. All subsequent ratios are expressed as a percentage of the time zero values.

FIG. 10. Effect of induced ATB0/ASCT2 antisense RNA expression on Na⁺-dependent glutamine uptake. Cultures of the SK-Hep parent cell line and SK-Hep cells stably transfected with a 1.3 kb ATB⁰ construct in the sense orientation (Sense 2-1) or antisense orientation (Antisense 1-1) were treated with inducer (MFP, 10 nM) or vehicle (ethanol, 0.1%) at time zero. At the indicated times thereafter, the initial rate (30 s) Na⁺-dependent transport velocity of 50 μM L-glutamine was measured by radiotracer analysis as described in the methods section. The results represent the average±SD of four separate determinations. *p<0.050 versus control. **p<0.010 versus control.

FIG. 11. Effect of induced antisense RNA expression and glutamine deprivation on cellular proliferation and viability. Cultures of SK-Hep cells stably transfected with a 1.3 kb ATB⁰ construct in the antisense orientation (Antisense 1-1) were treated at time zero with either inducer (FP, 10 nM), vehicle (ethanol, 0.1%) or maintained in the presence (+GLN) or absence (−GLN) of glutamine (DMEM+10% dialyzed FBS±2 mM L-GLN). At the indicated times thereafter, cell number was calculated after trypsmization and subsequent counting on a hemacytometer. The results represent the average±SD of five separate determinations. **p<0.010 versus-MFP. ††p<0.010 versus+GLN. Photographs were taken with a Kodak DC290 digital camera 48 h after induction. The arrows indicate cellular blebbing, a characteristic of apoptosis.

FIG. 12. Effect of antisense RNA expression on caspase-3 activation. Monolayer cultures of SK-Hep cells stably transfected with a 1.3 kb ATB⁰ construct in the sense orientation (S 2-1) or antisense orientation (AS 1-1) were treated at time zero with inducer MFP, 10 nM), vehicle (ethanol, 0.1%) or maintained in the presence (+GLN) or absence (−GLN) of glutamine (DMEM+10% dialyzed FBS±2 mM L-GLN). At the indicated times thereafter, cell lysates were obtained and the specific activity of caspase-3 was calculated as described in the methods section. The results represent the average of three separate determinations in one experiment. Similar results were obtained in two additional studies.

FIG. 13. TUNEL staining after induced antisense RNA expression. SK-Hep cells stably transfected with a 1.3 kb ATB⁰ construct in the antisense orientation (Antisense 1-1) were grown on FALCON® culture slides and treated±MFP for 24 h. After treatment, slides were washed in PBS, fixed in 4% paraformaldehyde and subjected to terminal deoxynucleotidyl transferase (TdT)-mediated dUTP nick-end labeling (TUNEL) staining as described in the methods section. Slides were observed under light microscopy and pictures were taken with a Kodak DC290 digital camera. A) −MFP, 100× B) +MFP, 100× C) +MFP, 400×, D) +MFP, 600×.

FIG. 14. Effect of induced antisense RNA expression and glutamine deprivation on the viability of multicellular tumor spheroids (MTS). Spheroids were generated in siliconized spinner flasks as described in the methods section. After the spheroids reached 500 μm in size (approximately 10 days), they were placed in 24-well plates and cultured±MFP or ±GLN for the indicated times. MTS and cells migrating from the surface as monolayers were observed by phase-contrast microscopy (PC), and assessed for viability by trypan blue exclusion (TB). Pictures were taken with a Kodak DC290 digital camera; original magnification, 40×.

FIG. 15. Panel A shows the relative effectiveness of each of the siRNA's in knocking down ASCT2 mRNA via northern analysis after 24 & 48 h of treatment. B is the phospho-imager quantification of the bands in A. C is the concentration curve for siRNA1, showing that 50 nM is sufficient for maximum silencing of ASCT2 expression.

FIG. 16. Panel A shows that siRNA1 (SEQ ID NO: 3) substantially decreases ASCT2 function (glutamine uptake) and, panel B shows that siRNA1 leads to cellular loss (death) compared to transfection reagent alone. Panel C are photos from the experiment of panel B.

FIG. 17. Panel A shows the effectiveness of siRNA1 at silencing ASCT2 expression in a panel of 6 human liver cancer cell lines (northern analysis). Panel B are the phospho-imager quantification results of the blot shown in panel A (normalized to beta actin mRNA levels).

### DETAILED DESCRIPTION OF THE INVENTION

The inventors have made the novel discovery that (1) hepatoma cells, liver tumor cells and cirrhotic liver exhibit increased expression of the ASC-mediated glutamine transport protein ATB⁰, and (2) the selective inhibition of ATB⁰ results in glutamine starvation and subsequent apoptosis of those cells, which exhibit increased expression of the ASC-mediated glutamine transport protein ATB⁰. It is envisioned by the inventors that the selective inhibition of ATB⁰ is useful in the treatment of hepatoma and other liver diseases. The inventors have also discovered that an siRNA (e.g., which comprise SEQ ID NO:3, 4, 5 or 6), effectively downregulates ATB⁰, with a concomitant death of hepatoma cells

The term “apoptosis” means programmed cell death as ordinarily understood by one skilled in the cell biological arts.

The term “agent” means any atom, ion, molecule, compound or chemical moiety, including, but not limited to a complex biological molecule such as an amino acid, peptide, polypeptide, protein, proteoglycan, carbohydrate, nucleic acid, polynucleotide, lipid, fatty acid, and steroid. A preferred agent inhibits the uptake of glutamine by a hepatoma cell, resulting in apoptosis of the hepatoma cell. A more preferred agent inhibits the glutamine transport activity of an amino acid transporter B⁰ (“ATB⁰” or “ASCT2”) protein. Most preferably, the agent is an antisense polynucleotide or siRNA, which inhibits or other wise blocks the translation of a polynucleotide encoding an ATB⁰ protein. The antisense polynucleotide or siRNA may be encoded by a DNA which is incorporated into a vector or otherwise operably linked to a promoter for inducible expression, temporal expression, tissue-specific or cell type-specific expression, or constitutive expression of the antisense polynucleotide. An “agent” may also be an inducing agent that induces expression of an ATB⁰-specificantisense or siRNA polynucleotide. For example, inducing agents include tetracycline, mifepristone, methanol, and the like. Vectors and other expression systems, such as adenovirus vectors and CMV promoter-driven systems, are well known in the art and readily available from commercial and non-commercial sources.

The phrase “uptake of glutamine” means the transport of glutamine from the environment outside of the cell, across the plasma membrane, and to the inside of the cell. The mechanism of uptake may include any means, including, but not limited to transport via System N, System ASC and ATB⁰.

The term “carcinoma cell” means any eukaryotic cell that has an unrestricted growth phenotype. A carcinoma cell is also a cancer cell, tumor cell, neoplastic cell or transformed cell.

The terms “heptocarcinoma”, “hepatocarcinoma cell”, “HCC”, “hepatoma” and “hepatoma cell”, which are equivalent in meaning, mean a liver cell that exhibits unrestricted growth or transformed phenotype. An hepatocarcinoma cell may reside anywhere within the body of an individual. An individual may be any vertebrate animal, preferably a mammal, more preferably a human.

The term “modulate” means to control the activity of a component of a biological pathway, and hence control that pathway to any extent. Modulate may be an increase or a decrease in activity relative to an activity that is not modulated. As used herein, an agent “modulates” a component of a glutamine transport system, by either increasing the activity of the component or decreasing the activity of the component, relative to the activity of the component in a similar cell that has not been contacted or treated with the agent. As used herein, “activity” means the biological function of a component of a biological pathway. For example, an activity of ATB⁰ is the transport of glutamine and other amino acids across the plasma membrane. Not meaning to limit the mode of modulation, it is envisioned that this activity may be modulated by increasing the rate of transport of glutamine by expressing a more active form of ATB⁰ in a cell, expressing more copies of ATB⁰ in the cell, decreasing the negative regulators of ATB⁰ in a cell, increasing the positive regulators of ATB⁰ in a cell, expressing a less active form of ATB⁰ in a cell, expressing fewer copies of ATB⁰ in the cell, decreasing the positive regulators of ATB⁰ in a cell, or increasing the positive regulators of ATB⁰ in a cell. In a further non-limiting example, the expression of a ATB⁰ antisense polynucleotide in a cell modulates a component of a glutamine transport system by decreasing the expression of ATB⁰ in a cell.

The phrase “glutamine transport system” means a system of biological “components”, such as transmembrane and other proteins, lipids, lipid and other moieties, ions such as sodium and calcium, ligands, and signaling molecules, which functions to transport glutamine fom outside of the cell, across the plasma membrane and into a cell. Examples of glutamine transport systems include system A, a sodium-dependent transport system which may comprise components ATA2 or ATA3, system N, a sodium-dependent transport system which may comprise components SN1 or SN2, system ASC, transport system which may comprise the component ATB0, and various other cation-dependent and independent systems that are known in the cell physiological arts. The term “component” means any biological component (supra). A preferred component is a transporter, of which ATB⁰ is an example.

The term “polynucleotide” means any polymer comprising at least two nucleotides joined together by way of a covalent phosphodiester bond. A polynucleotide may be a single stranded or double stranded DNA, RNA or hybrid DNA/RNA. A polynucleotide may comprise chemical modifications, such as but not limited to amino acid or other covalent adducts such as methyl groups and the like. A polynucleotide may be an “antisense polynucleotide”, which is a single stranded DNA or RNA that specifically associates with a sense RNA and inhibits or reduces the translation of the sense RNA into a polypeptide. An example of an antisense polynucleotide sequence is depicted in SEQ ID NO:1 and 2, which represent the reverse complement of ATB⁰ coding sequences. A polynucleotide may be a “ribozyme”, which is a molecule or ternary complex that comprises a catalytic RNA sequence, which may cleave a specific nucleotide sequence. A polynucleotide may be a “small interfering RNA” or “siRNA”, also known as double stranded RNA (“dsRNA”), micro RNA (“miRNA” or “micRNA). siRNAs are small RNAs of less than 50 nts long and usually 20-21 nts long. siRNAs are considered by one skilled in the art to be involved in the regulation of gene expression (gene silencing) and in the protection of a host genome against invasion by viral genomes, transposons or aberrant polynucleotides. It is demonstrated herein that an ATB⁰-specific siRNA would be effective in inhibiting ATB⁰ activity in a cell. Examples of such siRNAs comprise a sequence as set forth in any one of SEQ ID NO: 3, 4, 5 and 6.

The phrase “amino acid analog” means any molecule that can mimic the structure of a physiologically relevant amino acid and can bind to a protein that normally binds to the physiological relevant amino acid. For example, a glutamine-analog is expected to bind to a glutamine transporter with similar kinetics as glutamine binds to the glutamine transporter.

The term “antibody” means a complete antibody protein, a fragment of an antibody, a hybrid or chimeric molecule comprising at least a hyper variable region of a cognate antigen binding site of an antibody, or a Fab fragment. An antibody may be a population of antibodies (polyclonal) or monoclonal. An antibody demonstrates specific binding to an epitope and is useful in the identification of any object comprising the epitope, such as an antigen, and in the blocking of the activity of an object comprising the epitope. For example, it is envisioned that an antibody to ATB⁰ would modulate the activity of ATB⁰.

As used herein, the phrase “gene product” means any RNA transcribed from a gene or fragment of a gene, a cDNA, a polypeptide, a protein, or a fragment or modification thereof. For example, the meaning of ATB⁰ gene product includes an ATB⁰ transcript and ATB⁰ polypeptide.

The term “vector” means any vehicle for delivering an agent to a cell. A preferred vector comprises a polynucleotide that encodes an agent that modulates glutamine uptake, such as an ATB⁰-specific antisense or siRNA polynucleotide. Such a vector may be a virus-derived vector, such as an adenoviral, adenovirus-associated, lentivral, or other like vector, or a plasmid vector, such as an expression vector or inducible expression vector. A non-limiting example of an inducible vector is the pGene/V5-HisA vector, which is commercially available from Invitrogen Life Technologies as part of the GeneSwith™ Inducible Mammalian Expression System. Many vectors and expression systems are available for use in the practice of the invention and the skilled artisan may reasonably select an appropriate vector in the practice of the invention.

In one embodiment, the invention is drawn to methods of selectively killing a subset of liver cells by inducing apoptosis in the cells via the inhibition of glutamine uptake by the cells. The glutamine uptake is inhibited by treating the cells with an agent that inhibits the activity of a component of the ASC-mediated system of glutamine transport. In a preferred aspect of this embodiment, the agent specifically inhibits an ATB⁰ transporter, which is preferentially expressed in hepatoma cells and cirrhotic cells, which inhibits glutamine transport into the cells and causing the cells to undergo apoptosis. The cells may be in vivo or ex vivo.

Specific agents useful in the operation of this invention include antibodies, preferably antibodies that specifically bind to ATB⁰ and inhibit the uptake of glutamine through that transporter. Methods of making and testing antibodies, including both polyclonal and monoclonal, are well known in the art. Such methods are discussed at length in “Antibodies: A Laboratory Manual,” editors Harlow and Lane, Cold Spring Harbor Laboratory Press, 1999, which is incorporated herein in its entirety. It is envisioned that the ATB⁰-specific antibody is contacted to the cell by administering the antibody to the culture medium or to the individual orally or by injection. Given the fact that transporters are generally expressed on the cell surface and are generally exposed to the extracellular environment, antibodies are reasonably expected to be naturally directed to their specific targets upon administration to a patient or population of cells.

Specific agents that are useful in the practice of this invention include also polynucleotides, such as antisense polynucleotides and small interfering RNAs (siRNAs). Preferred antisense polynucleotides are specific to ATB⁰ RNAs, such as the sequences presented in SEQ ID NO:1 and 2. It is envisioned that fragments of those sequences, which are at least 10 nucleotides in length may be useful in the practice of this invention in the inhibition of ATB⁰ activity. Preferred siRNAs comprise sequences that include SEQ ID NO:3, 4, 5 and 6. An antisense polynucleotide or siRNA is preferably administered to a cell by contacting the cell with a vector that comprises a sequence that encodes the antisense polynucleotide or siRNA. The vector may be transferred into the cell by any means known in the art, such as via chemical transformation, electroporation or viral transduction. Preferred methods of getting the siRNA or antisense polynucleotide into the cell include, but are not limited to transfection using transfection reagents such as Lipofectamine™ (Invitrogen) or via viral transduction using virus vectors such as adenovirus. The skilled artisan, in the practice of this invention, would readily appreciate that many useful methods to get polynucleotides into a cell are applicable to the practice of this invention. Once in the cell, the encoding sequence is transcribed and the antisense polynucleotide or siRNA is transcribed and the antisense.

The agent of the instant invention, which is to be used in therapeutic or pharmaceutical compositions, can be administered by any suitable route known in the art including for example intravenous, subcutaneous, sublingual, intranasal, intramuscular, transdermal, intrathecal, transmucosal, pulmonary inhalation or intracerebral. Administration can be either rapid as by injection or over a period of time as by slow infusion or administration of a slow release formulation as in an, for example, an implantable gel or transdermal patch.

The agent of the instant invention can also be linked or conjugated with other materials that provide desirable pharmaceutical or pharmacodynamic properties. For example, the agent can be coupled to any substance known in the art to promote penetration or transport across the blood-brain barrier such as an antibody to the transferrin receptor, and administered by intravenous injection (See for example, Friden et al., Science 259:373-377, 1993). Furthermore, the agent can be stably linked to a polymer such as polyethylene glycol or fused to an albumin moiety to obtain desirable properties of solubility, stability, half-life and other pharmaceutically advantageous properties. (See for example Davis et al. Enzyme Eng 4: 169-73, 1978; Burnham, Am J Hosp Pharm 51: 210-218, 1994, which is herein incorporated by reference).

Preferably, the agent is administered with a carrier such as liposomes or polymers containing a targeting moiety to limit delivery of the agent to targeted cells. Examples of targeting moieties include but are not limited to antibodies, ligands or receptors to specific cell surface molecules.

For nonparenteral administration, the compositions can also include absorption enhancers which increase the pore size of the mucosal membrane. Such absorption enhancers include sodium deoxycholate, sodium glycocholate, dimethyl-β-cyclodextrin, lauroyl-1-lysophosphatidylcholine and other substances having structural similarities to the phospholipid domains of the mucosal membrane.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. The agent of the instant invention can also be incorporated into a solid or semi-solid biologically compatible matrix, which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing the agent are to be administered orally. Such formulations are preferably encapsulated and formulated with suitable carriers in solid dosage forms. Encapsulation helps to prevent the degradation of the agent in the digestive tract and may be employed to target the agent to the appropriate cell type. Encapsulation may, for example, be in the form of proteinoid microsphere carriers, as described in U.S. Pat. No. 4,925,673, which is incorporated herein by reference, or poly amino acid carriers as described in U.S. Pat. No. 6,242,495, which is incorporated herein by reference. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic or nucleic acid degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art based on the activity of the agent for a particular cell type ex vivo. The activity of a particular embodiment of the agent on cells in culture is described below and its activity on a particular target cell type can be determined by routine experimentation. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, the agent may be therapeutically administered by implanting into patients vectors or cells capable of producing a biologically-active form of the agent or a precursor of the agent that can be readily converted to a biological-active form of the agent by the body. For example, adenoviral vectors harboring a sequence that encodes an ATB⁰ antisense or siRNA may be administered to a patient by injection. It is well known in the art that those vectors accumulate in the liver and therefore bring the agent directly in contact with liver cells. The formulations and methods of this invention can be used for veterinary as well as human applications and the term “patient” as used herein is intended to include human and veterinary patients.

In yet another embodiment, the invention is directed to methods for diagnosing liver cancer in a patient. A patient may be any vertebrate, preferably a mammal, more preferably a human. The method comprises determining the amount of ATB⁰ in a sample obtained from the patient, relative to a standard, and determining the risk of liver cancer based upon a normal range. The sample may be serum or a tissue biopse, such as a liver biopse. Alternatively, the level of glutamine in the serum of the patient may be determined, compared to a standard, and the risk of liver cancer predicted on that basis.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### Example 1

### Selective Expression of ATB⁰ in Hepatocarcinoma Cells

### Materials and Methods

Cell Lines. The human hepatoma cell lines utilized in these studies were PLC/PRF/5 (34), SK-Hep (20), Hep3B (1) (American Type Culture Collection (ATCC), Rockville, Md.), Huh-7 (41) (from Dr. Jake Liang, Massachusetts General Hospital), FOCUS (28) (from Molecular Hepatology Laboratories, Massachusetts General Hospital Cancer Center) and the hepatoblastoma HepG2 (1), also from ATCC. A nontumorigenic human liver epithelial cell line termed THLE-5B was generated by immortalization with SV40 virus and was kindly provided by Dr. Curtis Harris at the National Cancer Institute (45). The JAR-1 human choriocarcinoma cell line from which the ATB⁰ cDNA was originally isolated was obtained from ATCC. All cells were maintained in Dulbecco's Modified Essential Medium (DMEM, 4.5 mg/ml D-glucose)+2 mM L-glutamine, 100 units/ml penicillin G, 100 μg/ml streptomycin and 10% FBS (all from Gibco/BRL, Gaithersburg, Md.).

Human Liver Tissue. The normal, cirrhotic and cancerous human liver tissue utilized for RNA analysis in these studies were from archived cryopreserved biopsies previously obtained from patients undergoing gastrointestinal surgery, as per the guidelines approved by the Massachusetts General Hospital Human Studies Committee and Institutional Review Board. Primary human hepatocytes were isolated as previously described in detail from freshly discarded pathological specimens (6). Primary human adult and fetal liver fibroblasts were isolated from primary cultures of liver cells after maintenance in the presence of 10% FBS. After 7-10 days, the fibroblasts overgrew the culture and were serially passaged by trypsinization. The fibroblasts were utilized between passages 3 and 10 in these studies. The primary hepatoblastoma sample was obtained from the Massachusetts General Hospital Tumor Bank. The fetal human liver RNA blot was kindly provided by Dr. David Rhoads. A commercially available human liver cancer RNA blot (Northern Territory™) was obtained from Invitrogen (Carlsbad, Calif.).

Glutamine Transport Assays. Measurement of initial-rate glutamine uptake was carried out via the cluster-tray method originally described by Gazzola (21) as reported previously (6, 9). Briefly, after trypsinization hepatoma cells were plated at a density of 1×10⁵ cells/well in 24-well culture plates (Costar Corp., Cambridge, Mass.) and allowed to grow to >80% confluence, typically one to two days later. For initial-rate measurements, the radiotracer utilized was L-[G-³H] glutamine (Amersham Corp., Arlington Heights, Ill.) at 4 μCi/ml, in the presence of unlabeled L-glutamine at 50 μM. For kinetic studies, the amount of unlabeled glutamine in the transport buffer varied from 10 μM to 10 mM. All transport measurements were carried out at 37° C. and were terminated after 30 s by three rapid washes with an ice-cold phosphate-buffered saline solution. Intracellular radiolabeled glutamine was extracted with 0.2 ml/well of 0.2% sodium dodecyl sulfate (SDS) and 0.2 N NaOH; after 1 h, 0.1 ml of the lysate was neutralized with 10 μl of 2 N HCl and subjected to scintillation spectrophotometry in a Packard TopCount™ (Packard Instruments, Meriden, Conn.). The remaining lysate was utilized for the determination of cellular protein by the bicinchoninic acid (BCA) method (Pierce Chemical Co., Rockford, Ill.). Rates of glutamine transport were calculated from the cpm per sample, the specific activity of the uptake mix (in cpm/nmol) and normalized to cellular protein content in a Microsoft Excel® spreadsheet program. Transport values obtained in the absence of extracellular Na⁺ (diffusion and Na⁺-independent uptake) were subtracted from those in the presence of Na⁺ (total uptake) to yield Na⁺-dependent rates which are reported in units of nmol•mg⁻¹ protein•30 s⁻¹. All transport values depicted are the average±standard deviation of four separate determinations. Kinetic analysis of Eadie-Hofstee linearized transport data (v vs. v/[L-GLN]) was performed by regression analysis (Cricket Graph®, Computer Associates, Islandia, N.Y.). Nonlinear regression analysis was performed with DataDesk (Data Description, Inc. Ithaca, N.Y.) and Excel for two component Michaelis-Menten kinetics:
v=((Vmax1×[S])/(Km1+[S])+(Vmax2×[S])/(Km2+[S])).

Glutamine-dependent Growth. Cells were plated in 96-well tissue culture plates (Falcon Labware, Franklin Lakes, N.J.) at a density of 2×10³ cells/cm². The following day, the cells were rinsed once and repleted with DMEM+10% dialyzed FBS (dFBS)+0, 0.05, 0.10, 0.15, 0.2, 0.4, 0.6, 0.8, 1.0 or 2.0 mM L-glutamine, with media changes every 48 h thereafter. In a subset of experiments designed to test the role of System ASC/B⁰-mediated glutamine uptake in cellular proliferation, cells were grown in DMEM+10% dFBS containing 0, 0.5 or 2.0 mM L-glutamine±a collective 20 mM excess System ASC substrates alanine serine and threonine (6.7 mM each). At specific times over 7 days, the relative cell number per well was determined by the MTT colorimetric assay (Sigma, St. Louis, Mo.) on a platereader (Anthos Labtec, Frederick, Md.) at a wavelength of 550 nm with a 650 nm reference filter. Relative growth rates were evaluated graphically with optical density as a function of time (days).

RNA Isolation and Northern Blot Procedure—Total cellular RNA was isolated from cultured cells or frozen tissue by the one-step acid-phenol guanidinium procedure (13) using Trisolve™ Reagent (13iotecx Corp., Houston, Tex.), subsequent treatment with RQ1 RNase-free DNase-I (Promega, Madison, Wis.), followed by an additional acid-phenol, phenol/chloroform/isoamyl alcohol, chloroform extraction and ethanol precipitation in the presence of sodium acetate. Equal amounts of total RNA (20 μg), as determined both spectrophotometrically and through ethidium bromide staining, were fractionated by electrophoresis through denaturing 1% agarose gels containing 0.2 M formaldehyde, transferred to nylon membranes by capillary action and UV cross-linked to the membrane.

The DNA templates utilized in this study were full-length human ATB⁰ ((hATB⁰); 2.9 kb EcoR1 insert (30)), human SN1 2.4 kb insert (19), and human ATA2 4.5 kb insert (25); all in pSPORT1 and kindly provided by Dr. Vadivel Ganapathy. Human albumin ((pLMALB); 1.8 kb EcoRI/HindIII fragment), alpha-fetoprotein ((pHAF7); 492 bp PstI fragment), IGF-II ((phins311); 8.6 kb EcoRI genomic DNA insert) and glutamine synthetase ((GS); HIBBA40, 2.7 kb HindE/NotI insert) were also utilized and were all obtained from ATCC. The inserts containing primarily coding sequence were excised from the plasmids with appropriate restriction enzymes, separated on agarose gels, excised, eluted and used as templates to generate α-³²P-dCTP labeled probes using a random primer labeling kit (Megaprime™, Amersham Corp., Arlington Heights, Ill.) according to the manufacturer's protocol. Hybridization with random hexamer-generated radiolabeled DNA probes was performed overnight at 65° C. in 5×SSPE with 7.5× Denhardt's reagent +0.5% SDS and 0.1 mg/ml sheared herring sperm DNA, after incubating the membrane for two hours under the same conditions.

Two of the DNA templates utilized in this study—human SN2 (42) and human ATA3 (23)—were generated by RT-PCR from human liver RNA, and engineered to contain SP6 sites (SEQ ID NO:7, 5′AATTTAGGTGACACTATAGA3′) in the 3′ termini for generation of riboprobe as described below. A 302 bp human SN2 cDNA representing bases 3-304 of the coding sequence was generated using a sense primer (SEQ ID NO:8, 5′GGAACTGCAGGATCCAAAGA3′) and antisense primer (SEQ ID NO:9, 5′AGGTCAGCAGGAGGTGGATG3′). Likewise, a 303 bp human ATA3 cDNA representing bases 1-303 of the coding sequence was generated using sense (SEQI ID NO:10, 5′ATGGATCCCATGGAACTGAG3′) and antisense (SEQ ID NO:11, 5′GGCCATGGCATAGGAC-AAGC3′) primers. Products of the expected size were obtained and verified by restriction endonuclease analysis. Riboprobes from PCR-generated and full-length cDNA's were generated using the Maxiscript SP6 kit from Ambion (Austin, Tex.) and α-³²P-UTP (Amersham Corp.). Ultra-Hyb (Ambion) at 72° C. was used in blots hybridized with riboprobe.

In all experiments, blots were washed under high stringency conditions (0.1×SSPE+0.5% SDS at hybridization temperature) and autoradiographic detection of the hybridization was achieved by exposure of Fuji Medical X-ray film at −80° C. In some experiments, the hybridized probe was stripped off the membrane by boiling in 0.1% SDS and the blots reutilized for the northern analyses of other genes. Where indicated, band intensities were quantified using the Kodak EDAS 290 system with 1D image analysis software (Eastman Kodak, New Haven, Conn.).

RT-PCR and Restriction Enzyme Analyses. ATB⁰ expression in individual cells and tissues was confirmed by RT-PCR and subsequent digestion with SalI or RsaI as described by Kekuda et al (31). The Perkin-Elmer GeneAmp® kit was utilized according to the manufacturer's instructions. The upstream primer was 5′CCGCTGATGATGAAGTCG3′ (SEQ ID NO:12) and the downstream primer was 5′CCCCCGATAGTGTTTGAG3′ (SEQ ID NO:13), which encompass nucleotides 1691-2197 of the hATB⁰ cDNA, yielding an expected amplification product of 507 bp. The RT-PCR reaction was carried out on RNA samples previously treated with RQ1 RNase-free DNase according to the manufacturer's instructions (Promega, Madison, Wis.). RNA (1 μg) was primed with oligo-dT, reverse transcribed, and subjected to 25 rounds of amplification (94° C./30 s, 60° C./30 s, 68° C./30 s for denaturation, annealing and extension steps, respectively). The RT-PCR products were isolated from the reaction by QIAquick PCR purification kits (Qiagen, Santa Clarita, Calif.) prior to analytical endonuclease digestion. The expected SalI digestion products were 277, 191 and 39 bp and those for RsaI are 330, 114 and 63 bp. All restriction enzymes were from Promega. The absence of reverse transcriptase in the reaction served as a negative control for all samples.

Statistical Analysis. Differences in specific measured parameters were evaluated for statistical significance by paired t-test (Microsoft Excel(®), and were considered significant when p<0.050.

### Results

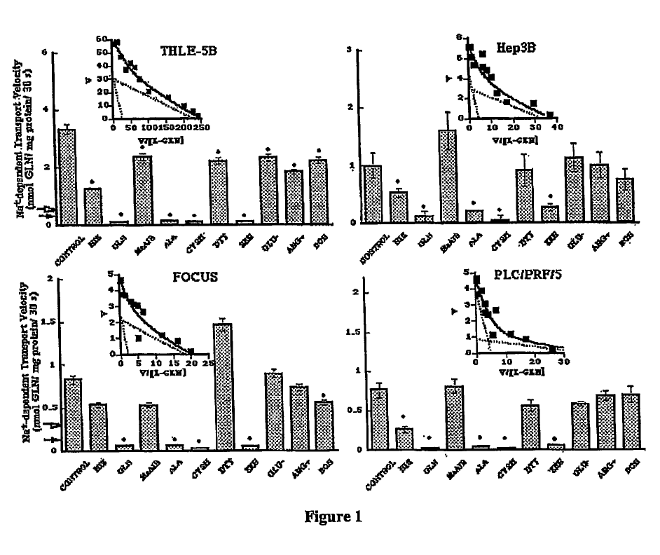

Glutamine Transport. In all cell lines examined, 90% or greater of glutamine uptake was Na⁺-dependent: Hep3B, 90%; FOCUS, 92%; PLC/PRF/5, 95%; THLE-5B, 99%. Initial rate Na⁺-dependent transport velocities for 50 μM L-glutamine were determined in the three additional human hepatoma cell lines and the results are shown in FIG. 1. Markedly enhanced rates (0.77 to 1.01 nmol•mg⁻¹ proteine•30 s⁻¹) were again observed compared to values for normal adult and fetal human hepatocytes (0.075-0.15 and 0.36-0.44 nmol•mg⁻¹ protein•30 s⁻¹, respectively (6)). Transport velocities were higher (p<0.010) in the nontumorigenic SV40-immortalized human liver epithelial THLE-5B cell line (3.33±0.16) versus the FOCUS (0.83±0.13), Hep3B (1.01±0.22) and PLC/PRF/5 (0.77±0.10 nmol•mg⁻¹ protein•30 s⁻¹). The glutamine concentration utilized in these assays not only allowed the measurement of initial-rate transport values, but also tested the ability of each line to take up low extracellular levels of this amino acid. When the transport of glutamine at physiological levels (500 μM) was measured in each of the lines, the results were qualitatively similar (data not shown).

FIG. 1 also shows that the expression of a System ASC-like transporter activity was largely responsible for the accelerated glutamine uptake in the additional four human cell lines, consistent with previous results obtained in the HepG2, SK-Hep and Huh-7 cell lines (6, 9). This conclusion is based upon significant (>80% to 90%) inhibition by alanine, serine and cysteine relative to all other amino acids tested, including the System A-specific substrate MeAIB, which failed to significantly inhibit glutamine uptake in all cell lines except the THLE-5B, where it diminished uptake by 29% (p<0.050). It should be noted that extensive depletion of intracellular amino acid pools were not performed prior to transport measurements in these studies because we sought to assess glutamine uptake under more physiological conditions. As a result, low affinity trans-inhibitable systems such as System A may not be operative or detectable under these conditions, even when expressed (see molecular analyses below).

Subsequent molecular studies (described below) revealed that more than one potential glutamine transporter was expressed in most cell lines, however. Therefore, two component nonlinear regression analyses were performed on the Na⁺-dependent transport kinetic data, where they were adequately resolved into high and low affinity components in all cell lines under study. The results are graphically depicted in FIG. 1 as insets. The Hep3B low affinity system exhibited a calculated Km of 1.0 mM and Vmax of 4.5 nmol•mg⁻¹ protein•30 s⁻¹, while the high affinity system had a derived Km of 90 μM and a Vmax of 3.0 nmol•mg⁻¹ protein•30 s⁻¹. The PLC/PRF/5 low affinity system was determined to possess a Km of 775 μM and Vmax of 3.9 nmol•mg⁻¹ protein•30 s⁻¹, while the high affinity system exhibited a derived Km of 23 μM and a Vmax of 0.82 nmol•mg⁻¹ protein•30 s⁻¹. For Focus, the low affinity system had a Km of 1.2 mM and a Vmax of 2.7 nmol•mg⁻¹ protein•30 s⁻¹, and the high affinity component possessed a Km of 122 μM with a Vmax of 2.2 nmol•mg⁻¹ protein•30 s⁻¹. For THLE-5B, the low affinity component exhibited a Km of 1.3 mM and a Vmax of 32.6 nmol•mg⁻¹ protein•30 s⁻¹, while the high affinity component had a Km of 135 μM and a Vmax of 30.8 nmol•mg⁻¹ protein•30 s⁻¹. Previous studies showed that the Huh-7 cell line took up glutamine primarily by System ASC based on amino acid inhibition profiles (9), so those results are not shown again. However, the kinetic analysis here revealed two resolvable components in this cell line with Km's of 50 μM and 861 μM and Vmax values of 7.3 and 11.7 nmol•mg⁻¹ protein•30 s⁻¹ for the high and low affinity components, respectively. The high affinity component in all cases is assumed to be System ASC based on the amino acid inhibition data and previously reported kinetic constants for this transporter (6, 9, 14, 15, 30, 31), while the low affinity components are more difficult to distinguish given that the System A & N isoforms isolated and characterized to date all possess similar affinities for glutamine in the high μM and low mM ranges (19, 23, 25, 42). Possibilities for each cell line will be offered discussion based on the molecular analyses described below.

Finally, based on the derived kinetic constants listed above, it was calculated that the high affinity component (System ASC) mediates greater than 80% of glutamine uptake at initial-rate concentrations (50 μM), and 60% -70% of uptake at physiological concentrations (600 μM) for all of the cell lines under study.

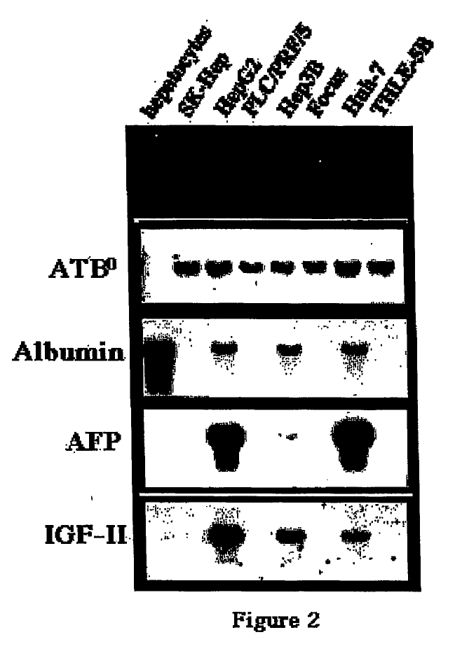

Northern Blot Analysis of ATB⁰: A cDNA encoding for a transporter with nearly identical characteristics as the System ASC activity described here was previously isolated from a human placental choriocarcinoma library (30). This gene was termed ATB⁰, for the transport activity designated as System B⁰, originally described in intestinal epithelial cells (49) and in renal epithelia (16) and otherwise identical to what has been termed System ASC in other cells and tissues (6, 9, 14, 15). Northern blot analysis with the human ATB⁰ cDNA-generated probe corroborated the results obtained in the amino acid inhibition profiles and kinetic studies (FIG. 2). A single mRNA species at approximately 2.9 kb was detected in all six human hepatoma lines under study and in THLE-5B, but not in normal human hepatocytes. The 2.9 kb mRNA size and lack of detectable expression in the liver are consistent with results obtained in the original isolation of this cDNA (30). Also illustrated in FIG. 2 is the independence of ATB⁰ expression from the differentiation state of the cell line, as both AFP- or albumin-positive and -negative cells express this transporter gene. Moreover, this transporter gene was expressed independent of IGF-II, an oncofetal hormone hypothesized to play a role in hepatocarcinogenesis (46).

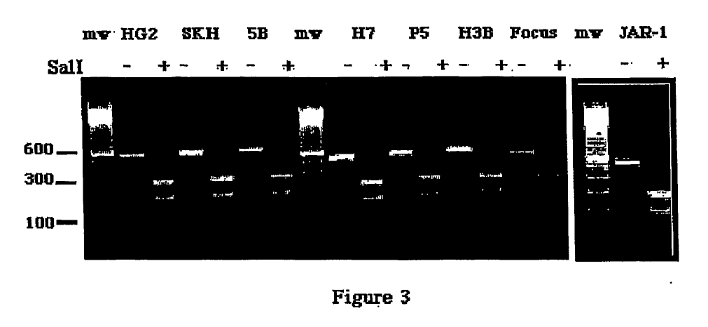

RT-PCR Analysis of ATB⁰ Expression: The RT-PCR analysis shown in FIG. 3 confirms that the 2.8 kb mRNA species obtained in northern blot analysis was indeed ATB⁰. RNA isolated from the six hepatoma cell lines, THLE-5B and JAR-1, (the choriocarcinoma cell line from which the ATB⁰ cDNA was isolated (30)) yielded the expected 507 bp RT-PCR product, which in turn produced the anticipated 277, 191 and 39 bp products upon digestion with SalI and 330, 114 and 63 bp products upon digestion with RsaI (not shown). When combined with the northern blot analyses, the data indicate that the ATB⁰ gene product probably underlies the relatively high System ASC glutamine uptake rates in human hepatoma and immortalized liver epithelial cells compared to adult and fetal hepatocytes. Accordingly, we refer to the cognate activity as “ASC/B⁰”-mediated transport.

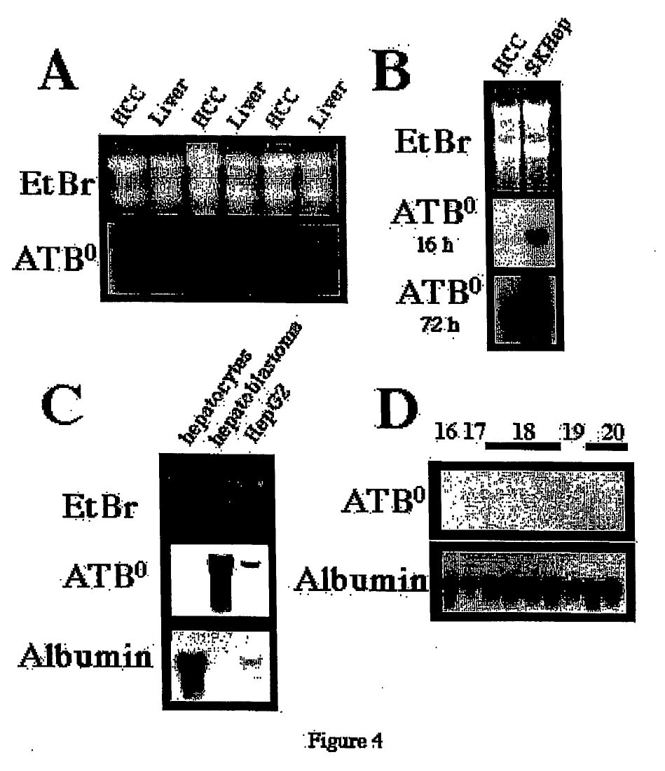

Expression of ATB⁰ in Clinical Liver and Liver Tumor Biopsies: All studies to this point had been carried out in human liver cancer cell lines, but we wanted to assess whether the ATB⁰ gene was expressed in human liver tumors in vivo. To this end, northern blot analysis was performed on total RNA from human liver and liver tumor biopsies. In a commercially available human liver tumor blot (Northern Territory™, Invitrogen) ATB⁰ mRNA was detectable in human hepatocellular carcinoma (HCC) samples, but not normal liver from the same patient in two out of three samples (FIG. 4A). To determine the relative level of ATB⁰ mRNA in HCC, a direct comparison was made with SK-Hep in a northern analysis. The expression levels of ATB⁰ mRNA in HCC were not as high as in SK-Hep cells (FIG. 4B), but in a hepatoblastoma biopsy this transporter gene was expressed at levels several-fold higher than in the hepatoblastoma cell line HepG2 (FIG. 4C). Expression of this glutamine transporter in the nontumorigenic SV40-immortalized THLE-5B liver epithelial cell line (FIGS. 1 & 2) raised the possibility that human liver cell growth alone may be sufficient for its transcriptional activation. However, ATB⁰ mRNA was undetectable in human fetal liver (FIG. 4D), corroborating previous results that showed fetal hepatocytes primarily utilize System N for glutamine uptake (6). Collectively, these results suggest that ATB⁰ mRNA is expressed at variable levels in adult and pediatric primary liver cancers, but the growth related signals that elicit its expression require further investigation.

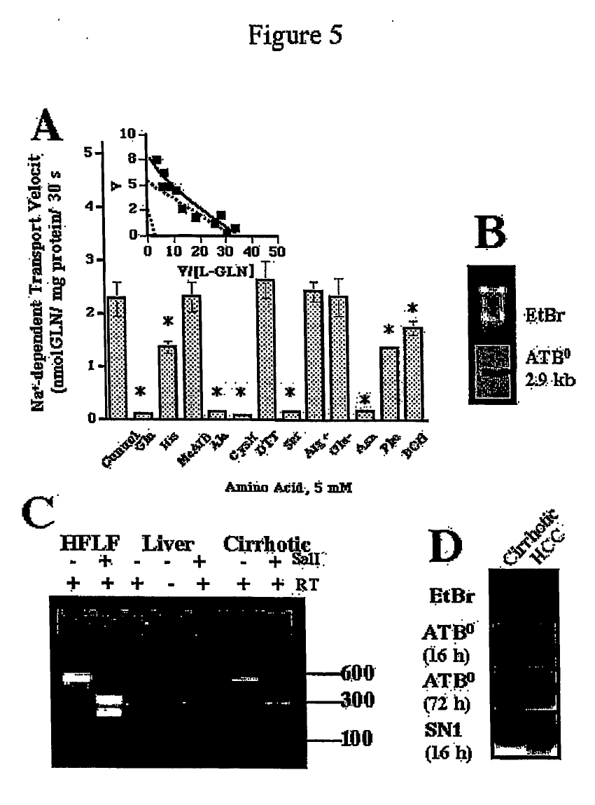

Expression of ATB⁰ in Fibroblasts and Cirrhotic Liver: Previously, it was believed that ATB⁰ expression was restricted to epithelial cells (16, 30). When glutamine uptake was characterized in human fetal liver-derived fibroblasts (HFLF), a profile nearly identical to that in the human hepatoma cells was observed (FIG. 5A), including 94% Na⁺-dependence, with marked inhibition by alanine, serine and cysteine, but not by MeAIB. Despite the well established expression of System A in human fibroblasts (22), the contribution of this transporter(s) to glutamine uptake does not become appreciable until extensive depletion of intracellular amino acids is performed (15)—conditions not utilized in this study. Nonlinear regression analysis of the Na⁺-dependent transport data yielded high affinity (Km=163 μM, Vmax=5.3 nmol•mg⁻¹ protein•30 s⁻¹) and low affinity (Km=1.2 mM, Vmax=3.1 nmol•mg⁻¹ protein•30 s⁻¹) components (FIG. 5A inset). The high affinity component, when combined with the amino acid inhibition data is consistent with what has been described as System ASC, while the low affinity component is assumed to be System A based on past studies with human fibroblasts (15, 17).

A single 2.9 kb ATB⁰ mRNA species was detectable in the fibroblasts with northern blot analysis (FIG. 5B). RT-PCR analysis confirmed the identity of the ATB⁰ mRNA in HFLF and cirrhotic liver, as evidenced by the 507 bp product and subsequent SalI digestion species of 277, 191 and 39 bp (FIG. 5C). When examined in biopsies from a patient with both liver cirrhosis and hepatocellular carcinoma (HCC), ATB⁰ mRNA was more abundant in the fibrous noncancerous tissue than in the tumor (FIG. 5D), further suggesting utilization of this transporter by mesenchymal cells. Glutamine transporter System N (SN1) mRNA was equally expressed in both samples, likely attributable to the presence of normal parenchymal epithelia in each (histology not shown), although in situ hybridizations will be required to definitively assign specific transporter expression to individual cell types in biopsies. These data suggest that the ATB⁰ gene product probably underlies “System ASC”—mediated glutamine uptake in fibroblasts, and that this transporter gene is expressed in mesenchymal as well as epithelial cells.

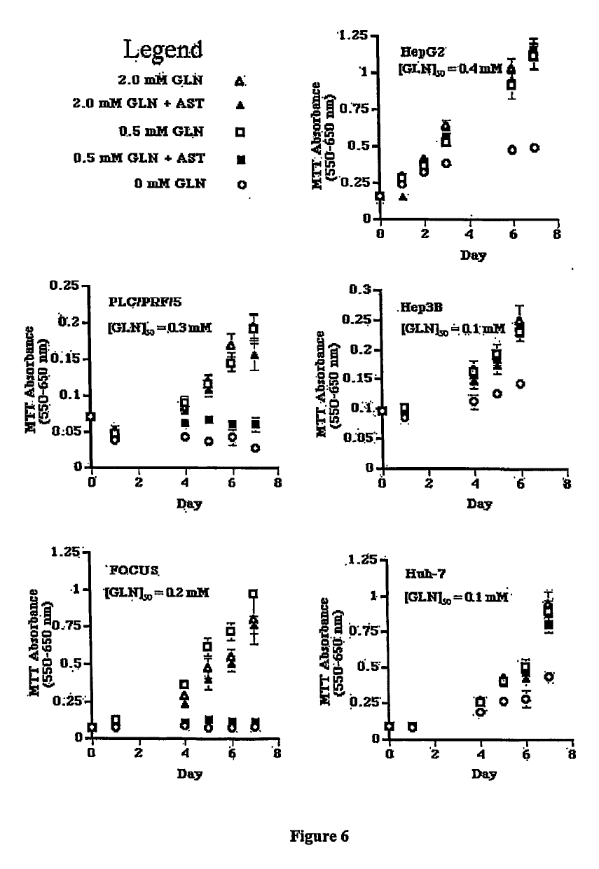

Role of ATB⁰ in Hepatoma Cell Proliferation: The essential role of glutamine in supporting the growth of cells in culture has been well accepted since the pioneering work of Harry Eagle (18). Previously, competitive inhibition of ASC/B⁰-mediated glutamine uptake in SK-Hep cells with excess alanine, serine and threonine in the culture media was shown to arrest growth (7). In order to assess the role of the ATB⁰ transporter in mediating glutamine-dependent growth in the additional five hepatoma cell lines, each was maintained in media containing specific concentrations of L-glutamine in the absence or presence of excess alanine, threonine and serine, (6.7 mM each, 20 mM collectively). While these amino acids are also System A substrates, there is no evidence for any MeAIB-inhibitable glutamine transport in these cell lines under the amino acid-rich conditions used in this study (FIG. 1). The results shown in FIG. 6 revealed that two of the hepatoma cell lines (PLC/PRF/5 and FOCUS) were likewise sensitive to substrate-dependent ASC/B⁰ inhibition in the presence of physiological levels (0.5 mM) of L-glutamine, whereas the Hep3B, HepG2 and Huh-7 were not. When the ambient L-glutamine concentration was raised to normal tissue culture levels of 2 mM, the negative effects of the three ASC/B⁰ substrates on growth were alleviated.

The individual responses to competitive transporter inhibition were unrelated to the relative glutamine requirements of each cell line for growth. The approximate concentration of glutamine that supported half-maximal growth (ED₅₀, in mM) for each cell line were as follows: sensitive lines: SK-Hep, 0.3 mM; FOCUS, 0.2 mM; and PLC/PRF/5, 0.3 mM; and refractory lines: Hep3B, 0.1 mM; HepG2, 0.4 mM; and Huh-7, 0.1 mM. Furthermore, the growth responses to competitive ASC/B⁰ substrate inhibition were independent of growth rates. The doubling times for the sensitive lines were: PLC/PRF/5, 53 h; FOCUS, 23 h and SK-Hep, 18 h. In the refractory lines doubling times were: Hep3B, 32 h; HepG2, 22 h and Huh-7, 20 h. It is interesting to note that the Hep3B, HepG2 and Huh-7 grow in the absence of glutamine—albeit at markedly attenuated rates—over the first week in culture (FIG. 6). When examined under light microscopy, however, the cells were clearly stressed (not shown). Nonetheless, the results indicate that three of six of the human hepatoma cell lines under study appear to largely rely upon the ASC/ATB⁰ transporter to meet the growth related demands for glutamine.

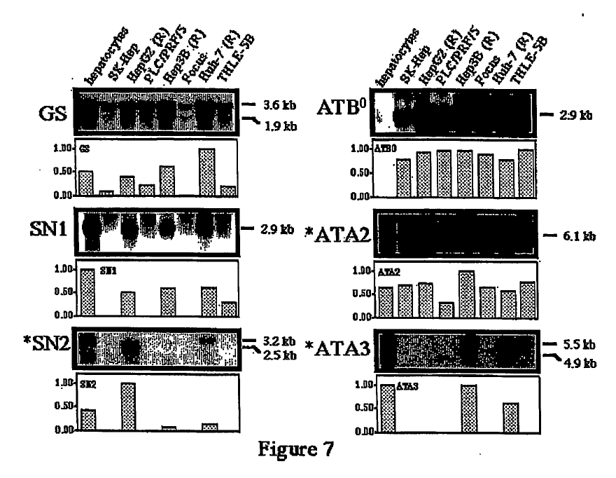

Northern Analysis of Glutamine Synthetase and Other Glutamine Transporters. A basis for the differential reliance upon the ASC/ATB⁰ transporter for growth in the hepatoma cell lines was provided by northern blot analysis of other glutamine transporters and glutamine synthetase. The expression of these potential compensatory genes was investigated as they might circumvent the effects of blunted glutamine uptake via ATB⁰ employed in the growth study. Despite relatively equal ATB⁰ mRNA expression, the hepatoma cells under study displayed a wide range of glutamine synthetase (GS), System N (SN1 & SN2) and System A (ATA2 & ATA3) isoform mRNA levels (FIG. 7).

GS mRNA was more abundant in the resistant hepatoma cell lines (Huh-7 (1.0)>Hep3B (0.62)>HepG2 (0.39)) versus the sensitive cell lines (PLC/PRF/5 (0.22)>SK-Hep (0.09)>Focus (0.02)), while SN1 mRNA was only evident in the resistant cell lines (Huh-7=Hep3B>HepG2). A 2.5 kb mRNA for System N transporter SN2 was markedly expressed only in HepG2, whereas a less abundant 3.2 kb SN2 mRNA species was expressed only in Huh-7 and Hep3B. Based on these results, it is possible that the Huh-7, HepG2 and Hep3B have a greater capacity to synthesize glutamine endogenously (from glutamate, ATP and ammonia) and are therefore less dependent upon transport for its supply. These three cell lines also apparently have the option of supplying cellular glutamine demands via System N-mediated transport.

The System A transporter ATA2 mRNA (6.1 kb) was expressed in all cell lines under study with Hep3B showing the highest expression levels (Hep3B>HepG2>SK-Hep>Focus>Huh-7>>PLC/PRF/5). In contrast, the mRNA for the ATA3 isoform was detectable only in the Hep3B (5.5 kb) and Huh-7 (5.5 and 4.9 kb) cell lines, even though this cDNA was originally isolated from a HepG2 cDNA library (23). The non-hepatoma THLE-5B cell line expressed only ATA2 in addition to ATB⁰, and possibly SN1 at very low levels, whereas normal human liver and/or hepatocytes expressed SN1, SN2, ATA2 and ATA3 isoforms at appreciable levels, but not ATB⁰ (FIG. 7). Thus, cell lines sensitive to ATB⁰-inhibited growth arrest (FIG. 6) express only ATA2 in addition to ATB⁰ for Na⁺-dependent glutamine uptake. Under the conditions used in this study, ATA2 would be trans-inhibited, rendering it of little compensatory use in the uptake of glutamine from the media when ATB⁰-mediated glutamine transport is competitively inhibited with excess substrates.

In summary, the northern analysis data indicate that the cell lines resistant to ATB⁰-targeted growth arrest and to some extent outright glutamine deprivation (HepG2, Hep3B and Huh-7, (FIG. 6)) have more compensatory mechanisms at their disposal for glutamine supply than do the poorly differentiated hepatomas (SK-Hep, Focus and PLC/PRF/5).

### Discussion

Given the potentially important role of glutamine in oncogenesis (39), the role of the liver and plasma membrane transport in glutamine homeostasis (27), and the observed depression of plasma glutamine levels in patients with liver cancer (29), the studies presented here were undertaken to determine whether a switch from System N to System ASC for glutamine uptake (6, 9) is a global or consistent feature of transformed human liver cells, and to identify the gene responsible for this accelerated activity. The results demonstrate that relatively high rates of System ASC-mediated glutamine transport are a consistent feature of all six human hepatoma lines studied to date, and that the product of the ATB⁰ gene—whose functional characteristics match those described for ASC-mediated glutamine uptake (6, 12, 30)—is probably responsible for this activity. This conclusion is based on marked inhibition of glutamine uptake by alanine, cysteine and serine relative to the other amino acids tested, including the System A-specific substrate MeAIB (FIG. 1) and the confirmed expression of this transporter via northern blot and RT-PCR (FIGS. 2 & 3). Although other transporters are expressed in some of the cell lines (FIG. 7), based on the kinetic analyses the high affinity component (ATB⁰) mediates greater than 80% of glutamine uptake at initial-rate concentrations (50 μM), and 60% -70% of uptake at physiological concentrations (600 μM) in the hepatomas and THLE-5B. An additional finding from these studies is that ATB⁰ is largely responsible for glutamine uptake in fibroblasts (FIG. 5). This is significant in that it was previously thought that ATB⁰ expression was restricted to epithelial cells (16, 31). Thus, the “System ASC” activity described in human fibroblasts for the past 20 years (12, 15, 22) is probably ATB⁰. The deduced amino acid sequence of human ATB⁰ gene is highly homologous (79% to 85% amino acid identity) to that encoded by rabbit ATB⁰ and mouse and rat ASCT2 (10, 31, 51). Based upon cross-species cDNA library screenings and nearly identical functional characteristics, it has been concluded that ASCT2 and ATB⁰ represent interspecies orthologous isoforms of the same transporter (5, 11). Thus, investigators might consider referring to this transporter as ASCT2 in all mammalian species. ATB⁰/ASCT2 is a member of the excitatory amino acid transporter (EAAT) family, which includes the glutamate transporters and another System ASC isoform (ASCT1) that does not transport the amides asparagine or glutamine (3, 48).

While it is clear that ATB⁰/ASCT2 mediates the majority of glutamine uptake in proliferating human liver-derived cells in vitro, signals for its transcriptional activation are poorly understood. Results in the immortalized nontumorigenic human liver epithelial cell line THLE-5B (45) suggest that its expression is not limited to tumor-derived liver cells (FIGS. 1, 2 & 3). In the original cloning and characterization paper, ATB⁰ was expressed in a number of normal human tissues, but its mRNA was not detectable in liver by northern blot analysis (30). The results in the present study corroborate those findings, as ATB⁰ mRNA is undetectable by northern blotting in isolated human hepatocytes, adult and fetal human liver (FIGS. 2 and 4)—cells and tissues that primarily utilize System N rather than System ASC/B⁰ for glutamine transport (6, 35). The lack of detectable mRNA and activity in fetal human liver (FIG. 4D and (6)) also indicates that liver cell growth per se is not alone sufficient to elicit ATB⁰ expression. Analysis of its expression in regenerating human liver would provide a better assessment of its activation as a result of hepatocyte cell cycle activation, but unfortunately no good source of regenerating human liver was available, with the possible exception of the cirrhotic liver sample in FIG. 5. In fact, no cellular system has yet been identified whereby ATB⁰ expression can be elicited from a previously dormant background, but previous work from our laboratory has shown a relationship between relative rates of ASC/B⁰-mediated glutamine transport activity and growth or growth-related processes. For example, chemically arrested hepatoma cells exhibit diminished glutamine uptake rates (9), while ASC/B⁰-mediated glutamine transport regulates DNA and protein synthetic rates in tumor cells (52). ASC/B⁰ activity has also been shown to fluctuate in a cell cycle-dependent manner in SK-Hep cells—approximately 40% higher in G1/S relative to G2/M (43). Recent work also showed that ASC/B⁰-mediated glutamine uptake increases threefold when SK-Hep cells are grown as large multicellular spheroids (44), and that chronic down-regulation of ASC/B⁰ activity with phorbol esters arrests the growth of this cell line (7). Despite these links between growth and glutamine transport activity, identification and study of the promoter region of the ATB⁰ gene will be required to address the mechanism of its activation in hepatoma and immortalized liver cells versus lack of expression in fetal liver.

To test the clinical relevance of our results obtained in human liver tumor cell lines, northern blot analysis showed that hepatocellular carcinomas expressed ATB⁰ mRNA, albeit at levels less than the HCC cell line SK-Hep (FIG. 4B). This could be the result of “dilution” of hepatoma RNA by normal hepatocyte RNA in the biopsy. It should be noted that the HCC samples shown in FIGS. 4B & 5D are from two separate patients, and contain approximately equal ATB⁰ mRNA levels. In contrast to HCC, the hepatoblastoma biopsy contained dramatically more ATB⁰ mRNA than the hepatoblastoma cell line HepG2 (FIG. 4C). However, it is difficult to assess whether relative mRNA levels directly correlate to ASC-mediated glutamine uptake rates. Clearly, a direct correlation between ATB⁰ mRNA levels and transport velocities was not apparent in the cell lines under study, as evidenced by the marked transport velocities in the THLE-5B cell line relative to the others (FIGS. 1 & 2). Such disparate activities in the face of comparable ATB⁰ mRNA levels further implicate significant translational and established posttranslational mechanisms (membrane potentials, intracellular amino acid levels (11, 31, 50)) in determining the measured rates of glutamine uptake via this carrier. Antibodies against the ATB⁰ protein, currently in development in inventor's laboratory, will help to resolve this issue in future studies, and will aid in the identification of ATB⁰-positive cells within tumor biopsies. Nonetheless, our results indicate that the ATB⁰ gene is expressed in liver tumors as well as liver tumor-derived cell lines.

With respect to the main finding in these studies of the ubiquity of ATB⁰ expression in hepatoma cells, why might a glutamine transporter switch from System N to System ASC/B⁰ be advantageous? The data presented in FIG. 6 suggest that some of the hepatoma cells rely more heavily on this transporter to drive glutamine-dependent growth than others with a greater capacity to produce glutamine endogenously or the ability take it up by alternative routes such as SN1 or SN2 (FIG. 7). This finding may be significant given the low GS activity observed in human liver tumors (37, 38). One practical consequence of ATB⁰ gene expression is that it allows cells to take up glutamine more efficiently than System N or A at relatively low ambient concentrations because of its higher affinity for this substrate (FIG. 1). A lower Km may prove effective in a poorly vascularized environment such as a tumor mass or connective tissue where glutamine concentrations may be diminished. Ultimately, different transport mechanisms between Systems N and ASC/B⁰ may underlie the heightened activity and preferential expression of the latter in human hepatoma cells. System ASC activity is known to be enhanced by “trans-stimulation”, a mechanism whereby increased levels of intracellular amino acid substrates accelerate the observed inward transport velocity of others (12, 22). ATB⁰ has recently been characterized by electrophysiological techniques and found to mediate Na⁺-dependent amino acid exchange rather than net uptake (50). Na⁺-dependent amino acid exchange allows cells to equilibrate pools of most zwitterionic amino acids via a “trade” of relatively abundant intracellular substrates for more coveted extracellular ones. Through this mechanism, ATB⁰ would indeed support growth and mediate the net uptake of glutamine, as faster growing hepatomas tend to have proportionally lower intracellular glutamine levels, at least in rodents (47), although no data are currently available on intracellular glutamine concentrations in the human hepatoma cell lines. Such an equilibrating mechanism may better lend itself to hepatoma growth than a narrow specificity carrier such as System N, whose properties are geared more toward systemic glutamine economy (5, 19). However, the mechanistic and cell specific basis for ATB⁰ gene activation must be elucidated before the teleological aspects of this problem can be soundly addressed.

To date, three isoforms of System A and two isoforms of System N have been identified, and all belong to the same transporter family (5). We examined the expression of both System N isoforms, and two of the three System A isoforms (ATA2 and ATA3) that are most liver-specific (23, 25). The ATA1 isoform appears to exhibit more brain-specific expression, although its mRNA has been detected in HepG2 cells (24), so its potential contribution to glutamine uptake in the cell lines under study here cannot be discounted. When the kinetic data are compared to the results with northern blots (FIG. 7), the low affinity component is probably ATA2 in the cell lines sensitive to ATB⁰ competitive growth inhibition (SK-Hep, PLC/PRF/5 and Focus (FIG. 6)), as no SN1, SN2 or ATA3 mRNA was detectable in these cells. Under the conditions utilized in the growth inhibition studies, ATA2 would be both trans-inhibited and competitively inhibited from taking up glutamine due to excess alanine, serine and threonine in the media, and would therefore serve little compensatory role in relieving the attenuated glutamine supply. In contrast, the cell lines resistant to ATB⁰ competitive growth inhibition (HepG2, Hep3B and Huh-7 (FIG. 6)) express both System N isoforms as well as ATA2, and ATA3 in the Hep3B and Huh-7. The low affinity component for Na⁺-dependent glutamine uptake is probably a collective contribution of the System A & N isoforms, as all exhibit deduced Km values for glutamine of 1-3 mM (19, 23, 25, 42). Alanine has recently been shown to be a marginal SN1 substrate (19), while serine has been shown to be a strong SN2 substrate (42), but neither amino acid has the capacity to completely block System N-mediated glutamine uptake. Moreover, the resistant cell lines grow at reduced rates for a brief period in the total absence of glutamine (FIG. 6); two of these three cell lines (Hep3B and Huh-7) also exhibited the lowest glutamine requirements for growth (ED₅₀=0.1 mM) suggesting a marginal glutamine metabolic economy in these cells. Under glutamine deprivation, intracellular glutamine could be provided by glutamine synthetase, whose mRNA is 2- to 50-fold more abundant in these cell lines relative to the three lines sensitive to ATB⁰ inhibition (FIGS. 6 & 7).

In summary, this study demonstrates that the ATB⁰ gene product largely mediates the accelerated rates of hepatoma cell line glutamine uptake reported here and previously (6, 9), as well as in human fibroblasts. The data also suggest that ASCT2/ATB⁰ may govern the growth of poorly differentiated human liver tumor-derived cells lacking System N expression and a limited capacity to produce glutamine endogenously, similar to HCC in vivo (37, 38). Based on the differential expression of ATB⁰ mRNA in the hepatocellular carcinoma samples and hepatoblastoma, it is possible that this transporter plays a role in the development and growth of certain liver tumors. Targeted molecular inhibition of ATB⁰ expression—work that is currently in progress in our laboratory—will provide further insights into the role of this glutamine transporter in hepatoma cell growth and survival.

Abbreviations used in this example are: HCC, hepatocellular carcinoma; SV40, simian virus-40; HFLF, human fetal liver fibroblasts; FBS, fetal bovine serum; dFBS, dialyzed fetal bovine serum; SSPE, Sodium chloride-Sodium Phosphate-EDTA solution; RT-PCR, reverse transcriptase-polymerase chain reaction; MeAIB, alpha-(methylamino)isobutyric acid; BCH, 2-amino-2-norbornane-carboxylic acid; MTT, 3-[4,5-Dimethylthiazol-2-yl]-2,5-diphenyltetrazolium bromide; ATB⁰, amino acid transporter B⁰; AFP, alpha-fetoprotein; IGF-II, insulin-like growth factor-two; GS, glutamine synthetase.

### Example 2

### Induction of Apoptosis of Hepatocarcinoma Cells

### Materials and Methods

Cell Culture. The human hepatoma cell line SK-Hepl (American Type Culture Collection (ATCC), Rockville, Md.) was maintained at 37° C. in a humidified atmosphere of 5% CO₂/95% air in Dulbecco's Modified Eagle Medium (DMEM, 4.5 mg/ml D-glucose) supplemented with 10% fetal bovine serum (FBS), 2 mM L-glutamine, 100 U/ml penicillin G and 100 μg/ml streptomycin (all from Invitrogen Life Technologies, Carlsbad, Calif.). SK-Hep cells stably transfected with both pSwitch and pGene/V5-HisATB⁰ (described below) were maintained in growth media supplemented with 300 μg/ml hygromycin B and 200 μg/ml Zeocin™ (both from Invitrogen Life Technologies). For glutamine deprivation studies, cells were grown in DMEM±2 mM L-glutamine, containing 10% dialyzed FBS (dFBS), 100 U/ml penicillin G, 100 μg/ml streptomycin and supplemented with hygromycin B and Zeocin™ in the case of stably transfected clones.

Inducible Antisense Expression System. The GeneSwitch™ inducible mammalian expression system (Invitrogen Life Technologies) was utilized for this study. The system involves a set of two vectors that are stably maintained by antibiotic selective pressure. The gene of interest is expressed in the pGene/5-HisA vector which contains a hybrid promoter sequence consisting of six binding sites for the yeast GAL4 protein and a 10 bp TATA box sequence from the Adenovirus E1b gene. Until induced, the promoter is transcriptionally silent allowing for extremely low levels of basal expression. The second vector, pSwitch, encodes for the GeneSwitch™ chimeric transcription factor that activates transcription from the pGene/V5-HisA vector containing the gene of interest. The GeneSwitch™ protein has three functional domains including: a GAL4 DNA binding domain, a human progesterone receptor ligand binding domain (hPR-LBD) that binds the inducing agent mifepristone (MFP) and an NFκB p65 activation domain to activate transcription from the GAL4 UAS/E1b minimal promoter of pSwitch. In the absence of MFP, the conformation of the HPR-LBD region prevents the GeneSwitch™ protein from activating transcription. MFP, also known as RU486, is a synthetic 19-norsteroid that normally acts as both a progesterone and glucocorticoid antagonist, but in this system acts as an agonist, binding the hPR-LBD region of the GeneSwitch™ protein inducing a transcriptionally active conformation.

Construction of the Inducible Antisense RNA Vector. The human ATB⁰ cDNA, kindly provided by Dr. Vadivel Ganapathy, was isolated from the pSPORT1 vector by a double restriction enzyme digestion using KpnI and NotI (both from Promega, Madison Wis.). The full-length ATB⁰ cDNA and the pGeneNV5-HisA vector were each subsequently digested at 37° C. overnight with ApaI (Promega). ApaI digestion of the ATB⁰ cDNA yields fragments of 107 bp, 170 bp, 218 bp, 569 bp and 1,333 bp. The 1,333 bp fragment of ATB⁰ represents base pairs 872-2205, spanning 82% of the coding sequence (base pairs 620-2245). ApaI-linearized pGene/V5-HisA vector was treated with shrimp alkaline phosphatase (Promega) followed by ligation with the gel-purified 1.3 kb ATB⁰ fragment at 4° C. overnight using T4 DNA Ligase (Promega). The resultant 5.9 kb pGeneN5-HisATB⁰ plasmid was subsequently transformed into E. coli competent cells, amplified and purified (Concert™ Maxiprep, Invitrogen Life Technologies). As only ApaI was used for the digestion, BamHI restriction digests of the 5.9 kb constructs were utilized to assess the orientation of any particular clone. Inserts were additionally confirmed by CEQ dye terminator cycle DNA sequencing (Beckman-Coulter, Fullerton, Calif.) with a Beckman CEQ 2000 capillary sequencer using primers specific for the pGeneN5-HisA multiple cloning site. These constructs, designated as pGene/V5-HisATB⁰ Antisense and pGene/V5-HisATB⁰ Sense, were used to generate stably transfected SK-Hep cells.

Generation of Stably Transfected Hepatoma Cells. SK-Hep cells were inoculated into 6-well plates (Costar, Cambridge, Mass.) at a concentration of 1×10⁵ cells/ml, allowed to adhere to the plates overnight, and were approximately 30% confluent for transfections the next day with Lipofectamine in OptiMEM serum-free media (both from Invitrogen Life Technologies), at a ratio of 8 μl Lipofectamine/μg of FspI-linearized pSwitch DNA. Transfections were performed by standard manufacturer-recommended procedures, and 48 h later the cells were removed by trypsinization and transferred into 150 mm tissue culture plates (Corning, Acton, Mass.). Cells were maintained in selective growth media supplemented with 300 μg/ml hygromycin B—a concentration empirically determined by titration analysis. Individual colonies were isolated 7 to 10 days later using sterile cloning cylinders (Sigma, St. Louis, Mo.) and maintained in selective growth media. A pGeneNV5-HislacZ reporter gene vector was used to identify individual colonies that exhibited the highest inducibility with the lowest levels of basal expression, via transient transfection and X-Gal (blue) staining. Appropriate stably transfected pSwitch SK-Hep clones were selected, one of which was utilized to generate the double stably transfected antisense and sense clones used in this study.

The pSwitch SK-Hep cells were transfected with either pGene/V5-HisATB⁰ Antisense or pGeneN5-HisATB⁰ Sense constructs as described previously. Colonies resistant to the pGeneNV5-His selectable marker Zeocin™ at an empirically determined concentration of 200 μg/ml were isolated. Several sense and antisense pGene/V5-HisATB⁰ clones were screened for their response to induction with MFP. All double stable transfected SK-Hep clones were maintained in selective media containing 300 μg/ml hygromycin B and 200 μg/ml Zeocin™.

RNA Isolation and Northern Blot Procedure. Total cellular RNA was isolated by the one step acid phenol—guanidinium method and analyzed by northern blotting analysis as previously described (57). Full-length 2.9 kb sense and antisense ATB^{0 32}P-labeled RNA riboprobes were generated by in vitro transcription from the ATB⁰ cDNA in the pSPORT-1 vector using a SP6/T7 MAXIscript™ kit (Ambion, Austin, Tex.) and α-³²P-UTP (Amersham Corp., Arlington Heights, Ill.). The pSPORT1 vector was linearized with HindIII (Promega) and transcribed with T7 RNA polymerase to generate the sense probe that detects induced antisense ATB⁰ RNA. Conversely, pSPORT1 was linearized with RsrII (New England Biolabs) and transcribed with the SP6 polymerase to make the antisense probe for detection of endogenous ATB⁰ mRNA. A 2.1 kb human ASCT1 cDNA [Shafqat, 1993 #1237] containing only coding sequence (kindly provided by Dr. Mike Kilberg) was excised from pcDNA3 with HindIII and XbaI and used to generate a random hexamer-primed radiolabeled probe (Megaprime™, Amersham) for northern analysis. A pTRI-Actin-Mouse DNA template (250 bp KpnI-XbaI fragment) was transcribed in vitro with the SP6 polymerase to generate a 334 bp antisense β-actin riboprobe as a positive control (Ambion). Membrane hybridization and washing under high stringency conditions was performed as previously described (57). Band intensities on X-ray film were quantified using the Kodak EDAS 290 system with 1D image analysis software (Eastman Kodak, New Haven, Conn.).

Glutamine Transport Assays. Measurement of initial-rate Na⁺-dependent glutamine uptake was performed by radiotracer analysis using the cluster tray method described in detail previously (57). Initial rate transport velocities are expressed as the average±SD of at least four separate determinations. The transport data was analyzed for significant differences using a t-test with a p<0.050 considered significant.

Cellular Quantification. Initial screens of the stably transfected sense and antisense ATB⁰/ASCT2 clones to MFP induction utilized the colorimetric MTT (3-[4,5-Dimethylthiazol-2-yl]-2,5-diphenyltetrazolium bromide (Sigma)) assay as previously described (57), using a spectrophotometric plate reader at a wavelength of 562 nm (Bio-Tek EL311SX, Winooski, Vt.). The absorbance is considered to be directly proportional to the number of metabolically active mitochondria, and by extension, the number of cells. In subsequent studies, cell number was quantified directly. At specific times after experimental treatment (±MFP or ±glutamine), cells were removed from the culture dish via trypsinization and quantified on a hemacytometer. In some cases, floating non-adherent cells were harvested, centrifuged briefly, and the resulting pellet resuspended in a smaller volume of fresh media. Cellular viability was assessed by trypan blue (0.04% w/vol) exclusion. Data was analyzed for significant differences by t-test with a p<0.050 considered significant.

Caspase Assays. Caspase-3 activity was analyzed with a chromogenic substrate (CaspACE™, Promega). Briefly, cells were plated in 100 mm plates (Corning) at a density of 5×10⁴ cells/ml, allowed to grow for 2 days, and were subsequently treated for 24 h in the presence of either 0.1% ethanol, MFP or MFP+Z-VAD-FMK (a pan-specific caspase inhibitor) or in the presence of GLN, −GLN or −GLN+Z-VAD-FMK. After the incubation, cells were lysed and protein extracts were collected. The protein content of each extract was determined by the bicinchoninic acid (BCA) method (Pierce Chemical Co., Rockford, Ill.) and an equal amount of protein from each sample was incubated with the caspase-3 substrate, Ac-DEVD-pNA. The amount of released p-nitroaniline (pNA) was analyzed by spectrophotometry at a wavelength of 405 nm. The specific activity of MFP-induced capase-3 activity was determined after normalization to both the ethanol and MFP+Z-VAD-FMK samples. The specific activity of GLN deprivation-induced caspase-3 activity was determined after normalization to both the +GLN and −GLN+Z-VAD-FMK samples.

TUNEL Staining. Terminal deoxynucleotidyl transferase (TdT)-mediated dUTP nick-end labeling (TUNEL) staining was performed with the DeadEnd™ colorimetric TUNEL system (Promega). Briefly, cells were plated on FALCON® culture slides (Becton Dickinson Labware, Franklin Lakes, N.J.) at a density of 5×10⁴ cells/ml and allowed to grow for 2 days, then treated ±MFP for 24 h. After treatment, slides were washed in PBS, fixed in 4% (w/vol) paraformaldehyde and stained according to the manufacturer's protocol. Biotinylated dUTP incorporated into nuclear DNA during the assay were reacted with streptavidin-conjugated to horseradish peroxidase (HRP). In the presence of H₂O₂, HRP converts the chromogen diaminobenzidine (DAB) to a stable dark brown product, allowing visualization of apoptotic cells.

Western Blot Analysis. Relative levels of intact and cleaved poly-(ADP-ribose) polymerase (PARP), total and phospho-specific double stranded RNA-dependent protein kinase (Protein kinase R (PKR)) were determined by western blot analysis using rabbit polyclonal antibodies directed against PARP, PKR and phosphothreonine (Thr 446/451) PKR (Cell Signaling Technology, Beverly, Mass.). Cellular lysates were prepared, separated by electrophoresis on 4-20% polyacrylamide gradient gels, transferred electrophoretically to PVDF membranes, and incubated with primary antibodies in blocking buffer (5% non-fat dry milk, 0.1% Tween-20 in Tris-buffered saline (TBS)) overnight at 4° C. Blots were washed three times in was buffer (0.1% Tween-20 in TBS). After a second incubation with a horseradish peroxidase (HRP)-linked anti-rabbit IgG antibody for 1 h, immunoreactive bands were visualized on X-ray film with a chemiluminescent HRP substrate (Phototope®, Cell Signaling Technology). The molecular size of the detected bands was calculated from molecular weight standards resolved on the same gels as the experimental samples. Band intensities on X-ray film were quantified using the Kodak EDAS 290 system with 1D image analysis software (Eastman Kodak).

### Results

Screening of Stably Transfected Hepatoma Cells. SK-Hep cells stably transfected with the inducible transcription factor (pSwitch) vector and with either pGeneNV5-HisATB⁰ Antisense or pGeneNV5-HisATB⁰ Sense (control) target constructs were screened initially for their growth response to MFP induction. The calorimetric MTT assay was utilized to quantify cell numbers of 12 antisense and 6 sense clones after induction with mifepristone (MFP), or treatment with vehicle (0.1% ethanol) for 48 h. While the screening process identified several highly responsive antisense clones that exhibited growth arrest after MFP induction, Antisense clone 1-1 was selected for further studies. Sense clone 2-1 was selected as a control, as it harbored the same insert as 1-1, but in the opposite orientation.

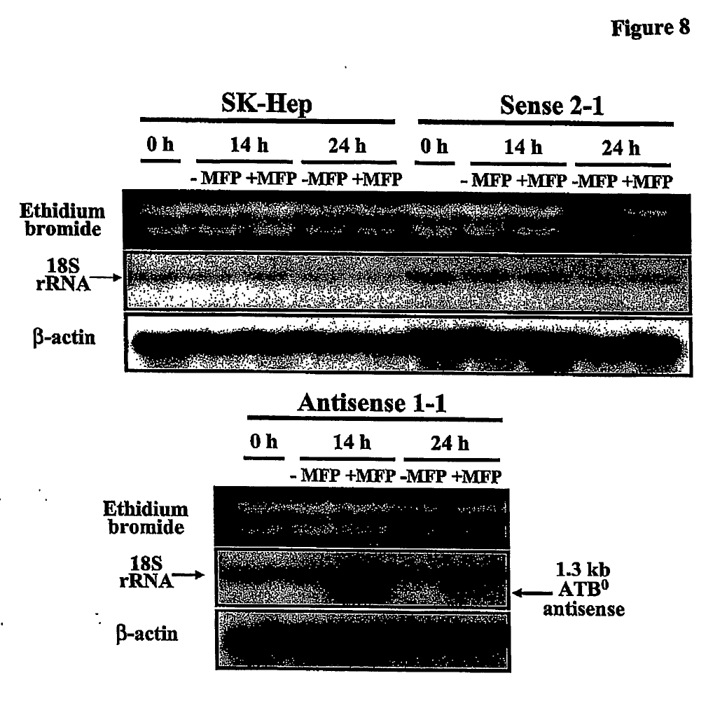

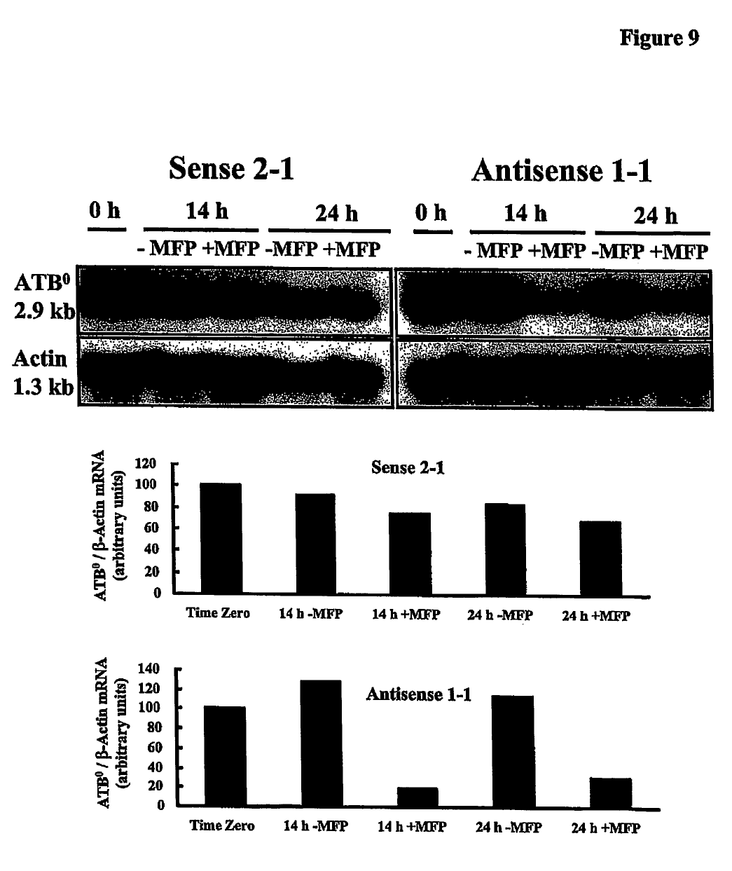

Effect of antisense expression on ATB⁰/ASCT2 mRNA levels. Upon induction with 10 nM MFP, a 1.3 kb antisense ATB⁰/ASCT2 RNA complementary to base pairs 872-2205 of the reading frame (620-2245) was detectable after 14 h in the Antisense 1-1 cells (FIG. 8), but by 24 h antisense RNA levels waned. Expression of the antisense RNA led to a decrease in cellular ATB⁰/ASCT2 mRNA levels of approximately 85% and 73% after 14 and 24 h, respectively, after normalization to β-actin levels in Antisense 1-1 (FIG. 9). This decrease was only observed in the Antisense 1-1 cells treated with MFP and not the vehicle control (0.1% ethanol). In contrast, Sense 2-1 cells treated with MFP or ethanol showed no change in endogenous ATB⁰/ASCT2 mRNA levels (FIG. 9). These results indicate that cells expressing the 1.3 kb antisense RNA exhibit markedly lower ATB⁰/ASCT2 mRNA levels compared to controls. To further investigate the specificity of the response, ASCT1 mRNA levels were also investigated. ASCT1 is 63% homologous to ASCT2 in the region targeted by the 1.3 kb antisense RNA. Although this ASC transporter isoform does not take up glutamine, it shares considerable substrate overlap with ASCT2 (3, 48). The results in FIG. 9 show that ASCT1 mRNA is markedly induced by ASCT2 knockdown after 14 and 24 h of antisense RNA induction.

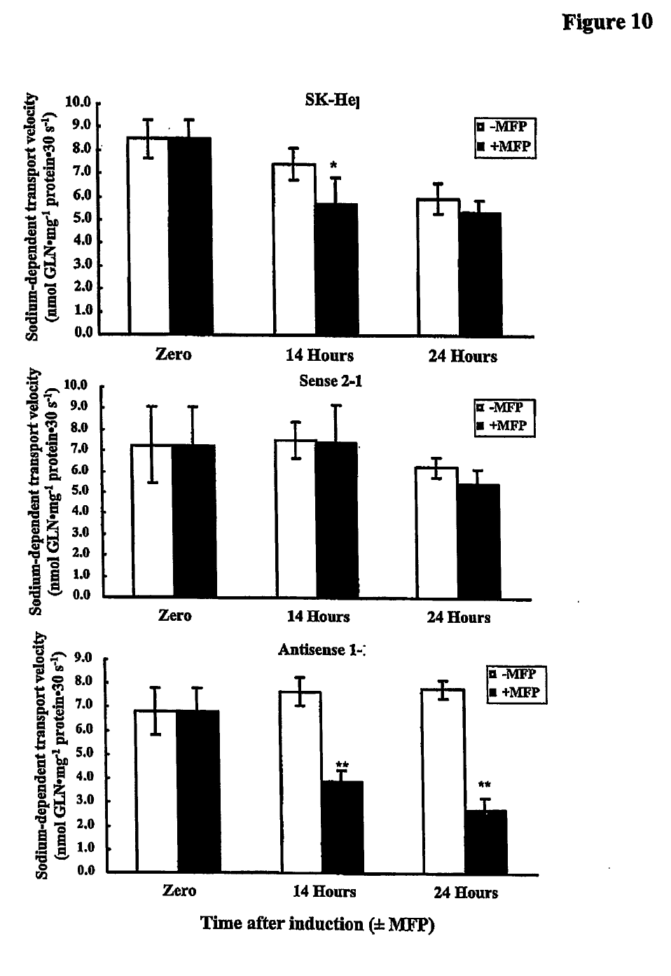

Inpact of antisense ATB⁰/ASCT2 RNA on glutamine transport rates. In order to determine the functional effect of targeting ATB⁰/ASCT2 mRNA, glutamine transport rates were measured. Sodium-dependent glutamine uptake rates revealed a significant (p<0.010) reduction of 49% and 65% in the Antisense 1-1 cells after induction with MFP for 14 and 24 h, respectively (FIG. 10). There were no significant differences in glutamine transport rates after induction for the Sense 2-1 cells; however, a slight but significant (p<0.050) 23% decrease was seen in the SK-Hep parent cells after 14 h of treatment with MFP (FIG. 10). This difference was not observed after 24 h (FIG. 10), and subsequent studies failed to reproduce this slight decrease at 14 h in the parent cells.

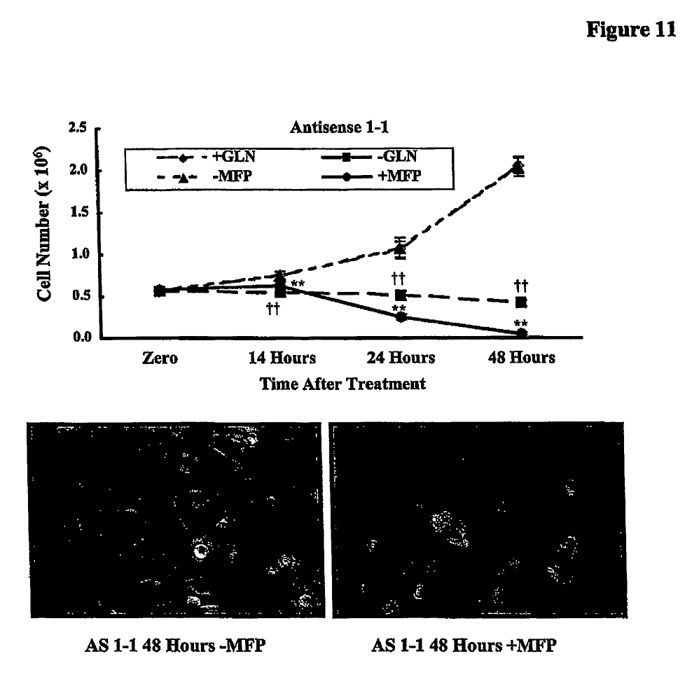

Effect of ATB⁰/ASCT2 antisense RNA on cellular growth and viability. Corresponding with the decreases in transporter mRNA levels and glutamine transport rates, the Antisense 1-1 cells also exhibited growth arrest with subsequent cellular loss. Significant (p<0.010) reductions in cell numbers of 17%, 77% and 98% were observed at 14, 24 and 48 h after induction, respectively, relative to controls (FIG. 11). In contrast, growth of the SK-Hep parent cell line and the Sense 2-1 controls was unaffected regardless of treatment with either MFP or 0.1% ethanol vehicle control (not shown).

Role of glutamine deprivation in antisense RNA-mediated cell death. The studies presented here evolved from the central role of ATB⁰/ASCT2 in driving glutamine-dependent growth of this liver cancer cell line (57,7). To determine what role glutamine deprivation plays in the cellular death of the Antisense 1-1 cells after induction with MFP, Antisense 1-1 cells were quantified after maintenance in glutamine-free media. Significant (p<0.010) reductions in cell numbers of 27%, 52% and 80% relative to controls were observed at 14, 24 and 48 h, respectively, after culture in the absence of glutamine (FIG. 11). These findings indicate that acute glutamine starvation causes a more rapid decrease in cell proliferation and viability than induced antisense RNA after 14 h (27% vs. 17%). However, after 24 and 48 h induced antisense RNA expression exerted a greater impact on cellular viability than glutamine deprivation (77% vs. 52% cell loss at 24 h and 98% vs. 80% cell loss at 48 h (FIG. 11)).

Characterization of hepatoma cell death. The reduction in cell number after induced ATB⁰/ASCT2 antisense RNA expression was attributed to cellular death and loss after the membrane integrity was compromised as determined by the lack of trypan blue exclusion (results not shown). The dead cells showed high levels of cell blebbing (FIG. 11), a hallmark of apoptosis, suggesting that the antisense RNA may elicit apoptosis. To examine this possibility further, we examined the activation of caspase-3, used terminal deoxynucleotidyl transferase (TdT)-mediated dUTP nick-end labeling (TUNEL) analysis to assay for DNA cleavage and examined PARP cleavage as well.

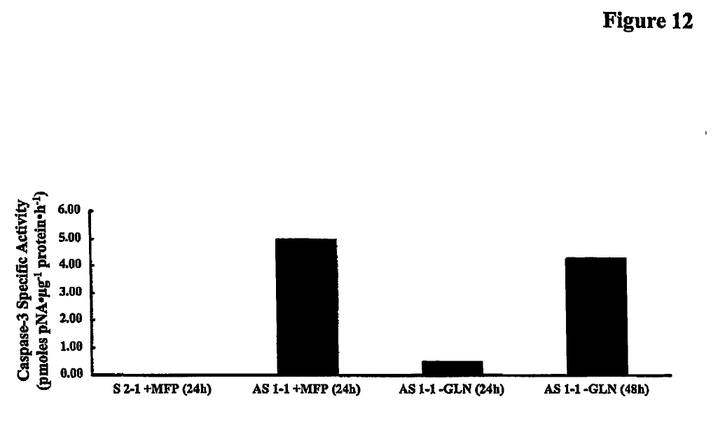

The effector caspase-3 has been shown to be active under a variety of conditions that elicit apoptosis. The specific activity of caspase-3 was 5-fold higher in Antisense 1-1 cells induced with MFP after normalization to the 0.1% ethanol treated controls (FIG. 12A). No significant differences in caspase-3 activity were observed between the Sense 2-1 cells treated with either MFP or 0.1% ethanol (FIG. 12). To determine the possible role of glutamine deprivation in apoptosis, caspase-3 activation was measured after maintaining the Antisense 1-1 cells in the presence or absence of glutamine. Caspase-3 specific activity showed a 50% increase after 24 h of glutamine deprivation, but a 4.3-fold increase was observed after 48 h (FIG. 12).

As nuclear fragmentation and caspase-dependent DNase activity is increased during apoptosis, we examined this endpoint via TUNEL staining in the Antisense 1-1 cells induced with MFP, since they showed a 5-fold increase in caspase-3 activation after 24 h of MFP induction. Antisense 1-1 cells cultured in the presence of MFP for 24 h showed marked levels of TUNEL-positive cells as compared to the vehicle controls (FIG. 12B). These TUNEL-positive cells exhibited several distinct circular locations of intense brown staining suggesting that the nuclei in these cells had been fragmented. In contrast, only a few random TUNEL-positive cells were seen in the SK-Hep parent cells and Sense 2-1 controls regardless of treatment with either MFP or ethanol (data not shown). Despite the positive TUNEL staining in MFP-treated Antisense 1-1 cells, laddering in genomic DNA was not clearly detectable by agarose gel electrophoresis and ethidium bromide staining (data not shown). (FIG. 12C).

Poly (ADP-ribose) polymerase (PARP) cleavage in experimental samples was assessed by western blot analysis. This DNA repair enzyme is one of the main targets of caspase-3, and is cleaved from its native molecular weight of 116 kDa to fragments of 89 and 24 kDa.

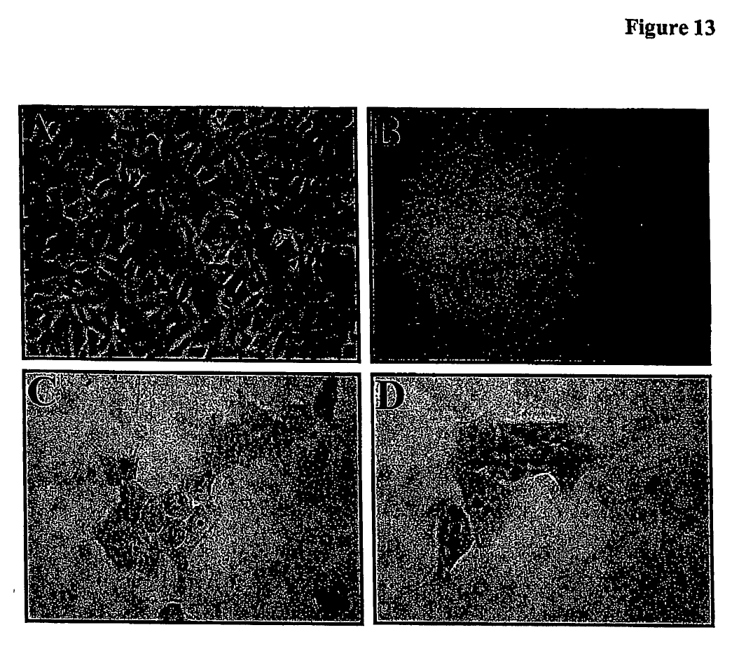

Possible role of a PKR response in hepatoma apoptosis. Given the marked effect of induced ATB⁰/ASCT2 antisense RNA expression on hepatoma viability and apoptosis, the possibility that the transporter knockdown response was a nonspecific double stranded RNA-dependent protein kinase (PKR) (62) mediated event was considered. To this end, cells were treated with Polyriboinosinic-polyribocytidylic acid (poly IC)—a mimic of double stranded RNA and stimulator of PKR in mammalian cells. The results shown in FIG. 13 demonstrate that poly IC failed to impact growth or viability of the SK-Hep cells. Moreover, when induced with MFP, Antisense 1-1 cells failed to exhibit enhanced PKR phosphorylation above baseline values, indicating that this enzyme is not activated by antisense RNA—mediated transporter knockdown. Taken together, these results indicate that the apoptosis elicited by ATB⁰/ASCT2 suppression is not attributable to a nonspecific antiviral response, but rather suggest an important role for this amino acid transporter in hepatoma growth and viability.

### Discussion

The studies presented here indicate that expression of the broad-scope Na⁺-dependent amino acid transporter ATB⁰/ASCT2 is essential for the growth and viability of SK-Hep human hepatoma cells, and that diminished glutamine delivery may contribute to the apoptotic cell death when transporter expression is inhibited. The liver normally serves as a systemic integrating center for glutamine homeostasis, but that role is subverted upon hepatocellular transformation, due to architectural, physiological and biochemical derangements (8). Previous work suggested that a high affinity “System ASC-like” activity underlies the 10- to 20-fold increase in glutamine uptake observed in human hepatoma cells relative to normal human hepatocytes (6). Subsequent studies revealed that the ATB⁰ mRNA (30), also known as ASCT2 (58), encodes for this activity, and that competitive inhibition of glutamine uptake with other System ASC substrates arrested growth in three hepatoma cell lines (SK-Hep, Focus and PLC/PRF/5)(7,57). ATB⁰/ASCT2 is expressed in every proliferating human liver-derived cell, hepatoma cell line and primary liver cancer biopsy examined, but it is not expressed in normal human hepatocytes (57). Collectively, these observations led to the hypothesis that this transporter might serve as a selectively targeted therapy for HCC, and that inhibition of its expression would lead to hepatoma growth arrest or apoptosis. The results presented in this example provide a proof-of-concept for that hypothesis, demonstrating that an aggressively growing human HCC cell undergoes apoptosis within 48 h after antisense RNA-mediated attenuation of ATB⁰/ASCT2 expression.

Antisense oligonucleotides have been successfully used to target glutamate transporters (63, 64) that belong to the same excitatory amino acid transporter (EAAT) family as ATB⁰/ASCT2 [Kanai, 1997 #2249]. Initially, several clones stably transfected with ATB⁰/ASCT2 antisense constructs were isolated, and most showed the same temporal kinetics of cell death after MFP induction as clone Antisense 1-1, which was selected for the more detailed studies presented in this report. Expression of the 1.3 kb antisense RNA elicited a significant decrease in endogenous ATB⁰/ASCT2 mRNA levels and sodium-dependent glutamine uptake rates at 14 and 24 h after induction (FIGS. 2 & 3). It is difficult to assess the effects of antisense RNA on transporter protein turnover, as antibodies against this transporter that are useful in immunoprecipitation or western blot analyses have proven difficult to produce. Instead, glutamine uptake was measured, given that ATB⁰/ASCT2 mediates over 90% of glutamine uptake into SK-Hep cells at the initial-rate concentrations (50 μM) used in the transport assays (6).

The results also indicate that induced ATB⁰/ASCT2 antisense RNA expression exerts a more rapid effect on cell growth and viability than overt glutamine deprivation (FIGS. 11, 12 and 14). After 48 h of induced antisense expression, there was a 98% reduction in cell number compared to an 80% reduction in cell number after glutamine deprivation. The loss of cells observed after glutamine deprivation and induction of ATB⁰/ASCT2 antisense RNA are both attributed mainly to apoptosis as assessed by caspase-3 activation (FIG. 12A). Given the glutamine dependence of rapidly proliferating cells, and the fact that ATB⁰/ASCT2 mediates 70%-80% of glutamine uptake in this cell line at tissue culture levels (2 mM) (57), we originally hypothesized that insufficient glutamine supply underlied the bulk of the apoptotic response to transporter knockout. However, ATB⁰/ASCT2 mediates the transport of a number of other zwitterionic amino acids in addition to glutamine (12). Specifically, it has been shown to be a Na⁺-dependent amino acid exchanger, mediating the transmembrane exchange of relatively abundant intracellular zwitterionic amino acids for more coveted extracellular substrates. In this capacity ATB⁰/ASCT2 has been proposed to equilibrate cytoplasmic amino acid pools (50). The data suggest that disruption of this homeostatic function may synergize with suppression of glutamine delivery to more rapidly elicit apoptosis in SK-Hep cells.

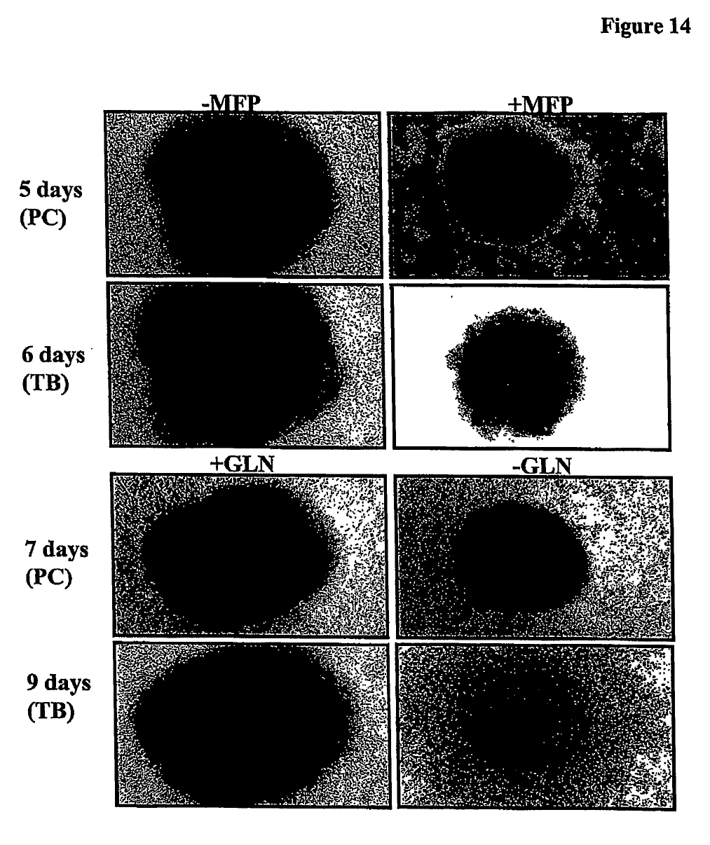

The mechanism leading from ATB⁰/ASCT2 inhibition to apoptosis is unclear, but is currently being investigated. As we ruled out a nonspecific double stranded RNA (PKR) response (FIG. 14), we hypothesize that another cellular stress pathway links transporter suppression to apoptosis. Specifically, oxidative stress may be involved due to the likely loss of the antioxidant glutathione after diminution of its precursor glutamine (65, 66). PKR phosphorylates the α chain of the eukaryotic translation initiation factor eIF2 inhibiting the guanine nucleotide exchange factor eIF2B and turning off protein synthesis [62, 67]. In this respect, the selective expression of ATB⁰/ASCT2 in cancerous versus normal human liver cells remains important since tumor-specific activation of PKR has recently been used to inhibit glioma growth in vivo [68]. However, treatment of SK-Hep cells with polyinosinic-polycytidylic acid (poly-IC), a potent and specific elicitor of the PKR response [67] failed to elicit the apoptotic response observed with the inducible antisense RNA. Interestingly, another member of the eIF2α kinase family [69, 70], GCN2, has been shown to be activated during amino acid starvation in yeast [71] and its mammalian ortholog has recently been identified [72, 73]. Studies are currently underway to examine these pathways, and their possible role, if any, in eliciting apoptosis in response to suppressed ATB⁰/ASCT2 expression.

In conclusion, the studies presented here provide initial evidence that attenuating ATB⁰/ASCT2 expression leads to apoptotic cell death of an aggressively growing human liver cancer line. Given that ATB⁰/ASCT2 is not expressed in normal hepatocytes, these results suggest that this transporter is a reasonable selective target within the liver for treatment of HCC.

### Example 3

### siRNAs Modulate ASCT2 Activity of Hepatoma Cells

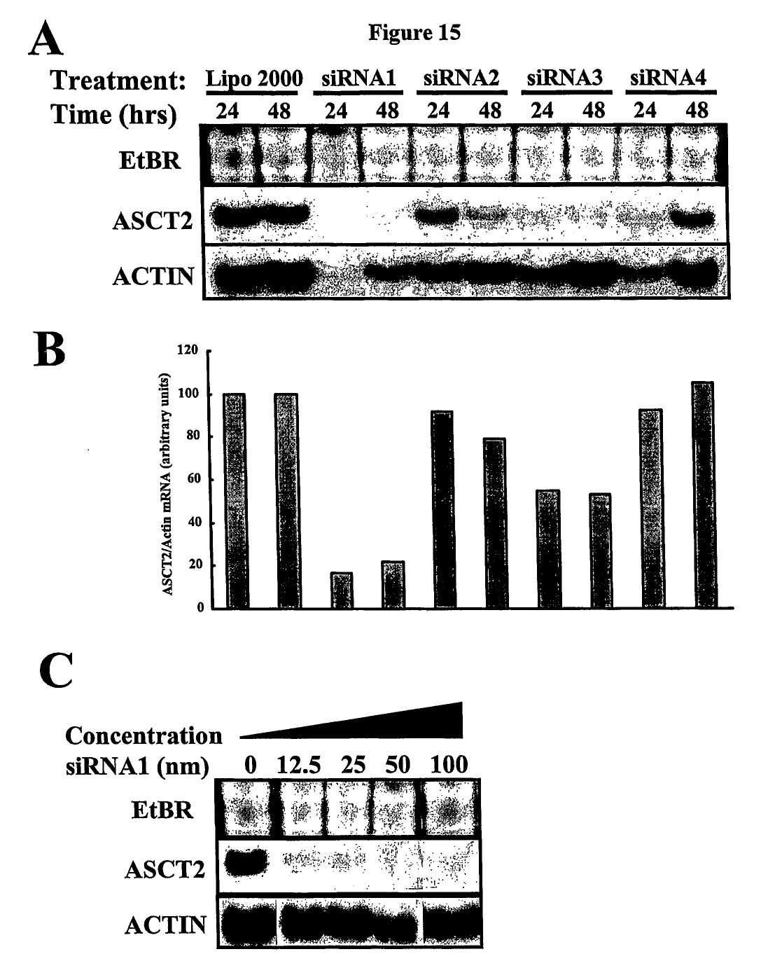

siRNAs that are specific to ASCT2 were designed using several known algorithms and then checked for specificity. Four (4) siRNAs, i.e., siRNA1 (which comprises SEQ ID NO:3), siRNA2 (which comprises SEQ ID NO:4), siRNA3 (which comprises SEQ ID NO:5), and siRNA4 (which comprises SEQ ID NO:6), were synthesized. SK-Hep cells were transfected with 50 nM concentration of any one of siRNA1, 2, 3 or 4 using Lipofectamine™ 2000 (Invitrogen™ life technologies) based on the protocols set forth in Gitlin, L., Karelsky, S., and Andino, R. (2002), Nature 418: 430-434; Yu, J. Y., DeRuiter, S. L., and Turner, D. L. (2002), Proc. Nat. Acad. Sci. USA 99:6047-6052; and “Transfecting siRNA into Mammalian Cells Using Lipofectamine™ 2000,” Form No. 18057N, Doc. Rev. 102802 by Invitrogen Corporation (2002), which are herein incorporated by reference. At 24 hours and at 48 hours, RNAs were extracted from the transfected cells and the expression of ASCT2 was examined by Northern blot analysis (see FIG. 15A). ASCT2 mRNA expression relative to actin mRNA levels was quantified using a phosphorescence imaging system (FIG. 15B), which demonstrated that siRNA1 (SEQ ID NO:3) appeared to be the most effective of the four siRNAs at reducing ASCT2 expression in hepatoma cells.

Hepatoma cells were then treated with Lipofectamine™ 200 without siRNA1, with 12.5 nM, 25 nM, 50 nM or 100 nM siRNA1. 50 nM siRNA1 was observed to be sufficient for maximum silencing of ASCT2 expression (FIG. 15, panel C). siRNA1 was used in all subsequent experiments.

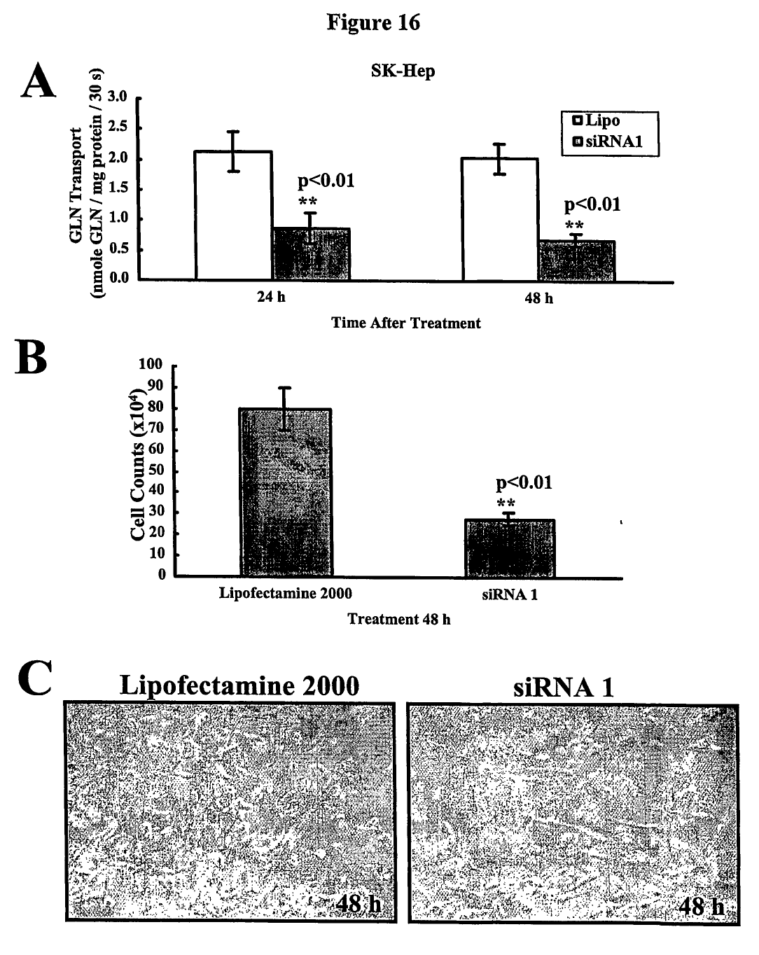

The physiological effects of downregulating ASCT2 expression by administration of siRNA1 in hepatoma cells were studied. Glutamine transport, which was measured as nanomoles of glutamine per milligram of total protein per 30s, was examined at 24 hours and at 48 hours in Lipofectamine treated cells and Lipofectamine+50 nM siRNA1. Those cells treated with siRNA1 exhibited greater than 50% reduction (p<0.01) in glutamine transport relative to controls (FIG. 16, panel A). Cell counts were made at 48 hours of hepatoma cultures, each of which were started with the same number of viable cells, receiving either Lipofectamine™ 200 alone or Lipofectamine™ 200 plus siRNA1. After 48 hours, a 40% reduction in the number of cells that had received isRNA1 (which comprises SEQ ID NO:3) relative to control cells was observed. This indicates that siRNA1, which blocks expression of ASCT2/ATB⁰, leads to increased cell death of hepatoma cells.

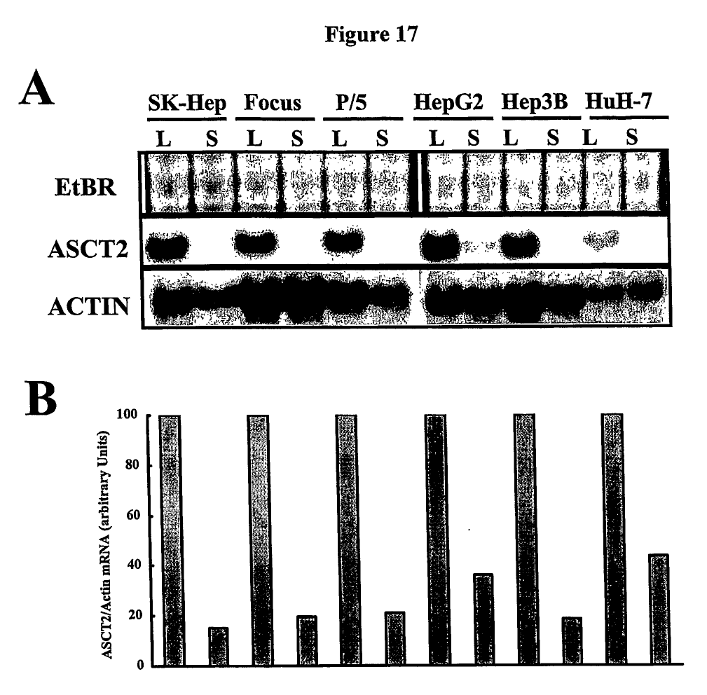

The effectiveness of siRNA1 at silencing ASCT2/ATB⁰ is shown in FIG. 17. RNA was extracted from six (6) human liver cancer cell lines and subject to northern blot analysis using ASCT2/ATB⁰ DNA as a probe. Actin RNA was probed as a baseline control. FIG. 17, panel A shows the ethidium bromide stained gel and the autoradiogram of ASCT2/ATB⁰ RNA hybridization and actin RNA hybridization. Those autoradiograms were subjected to phospho-imager quantitative analysis and normalized to actin RNA expression (FIG. 17, panel B). The designation “L” refers to Liopfectamine™ 200 alone, and “S” refers to Liopfectamine™ 200 plus siRNA at 50 nM. A significant reduction in relative ASCT2.ATB⁰ expression was observed in those hepatoma cells that had received siRNA1.

3B are the quantification results of the blot shown in 2A

(normalized to beta actin mRNA levels).

### References

The following references are cited above using the associated numerical identifiers. Applicant makes no statement, inferred or direct, regarding the status of these references as prior art. These references are incorporated herein by reference.

1. Aden, D, Fogel A, Plotkin S, Damjanov I, and Knowles B. Controlled synthesis of HBsAg in a differentiated human liver carcinoma-derived cell line. Nature 282: 615-616, 1979.

2. Ahluwalia, G S, Grem J L, Hao Z, and Cooney D A. Metabolism and action of amino acid analog anti-cancer agents. Pharmacol Ther 46: 243-271, 1990.

3. Arriza, J L, Kavanaugh M P, Fairman W A, Wu Y N, Murdoch G H, North R A, and Amara S G. Cloning and expression of a human neutral amino acid transporter with structural similarity to the glutamate transporter gene family. J Biol Chem 268: 15329-15332, 1993.

4. Baggetto, L G. Deviant energetic metabolism of glycolytic cancer cells. Biochimie 74: 959-974, 1992.

5. Bode, B P. Recent molecular advances in mammalian glutamine transport. J Nutr 131, Suppl. 4S: 2475S-2487S, 2001.

6. Bode, B P, Kaminski D L, Souba W W, and Li A P. Glutamine transport in isolated human hepatocytes and transformed liver cells. Hepatology 21: 511-520, 1995.

7. Bode, B P, Reuter N, Conroy J L, and Souba W W. Protein linase C regulates nutrient uptake and growth in hepatoma cells. Surgery 124: 260-268, 1998.

8. Bode, B P, and Souba W W. Glutamine transport and human hepatocellular transformation. J Parent Enteral Nutr 23: S33-37, 1999.

9. Bode, B P, and Souba W W. Modulation of cellular proliferation alters glutamine transport and metabolism in human hepatoma cells. Ann Surg 220: 411-424, 1994.

10. Broer, A, Brookes N, Ganapathy V, Dimmer K S, Wagner C A, Lang F, and Broer S. The astroglial ASCT2 amino acid transporter as a mediator of glutamine efflux. J Neurochem 73: 2184-2194, 1999.

11. Broer, A, Wagner C, Lang F, and Broer S. Neutral amino acid transporter ASCT2 displays substrate-induced Na⁺ exchange and a substrate-gated anion conductance. Biochem J 346: 705-710, 2000.

12. Bussolati, O, Laris P C, Rotoli B M, Dall'Asta V, and Gazzola G C. Transport system ASC for neutral amino acids. An electroneutral sodium/amino acid cotransport sensitive to the membrane potential. J Biol Chem 267: 8330-8335, 1992.

13. Chomcynski, P, and Sacchi N. Single-step method of RNA isolation by acid guanidinium thiocyanate-phenol-chloroform extraction. Anal Biochem 162: 156-159, 1987.

14. Collins, C L, Wasa M, Souba W W, and Abcouwer S F. Determinants of glutamine dependence and utilization by normal and tumor-derived breast cell lines. J Cell Physiol 176: 166-178, 1998.

15. Dall'Asta, V, Rossi P A, Bussolati O, Guidotti G G, and Gazzola G C. The transport of L-glutamine into cultured human fibroblasts. Biochim Biophys Acta 1052: 106-112, 1990.

16. Doyle, F A, and McGivan J D. The bovine renal epithelial cell line NBL-1 expresses a broad-specificity Na⁺-dependent amino acid transport system (system B⁰) similar to that in bovine renal brush border membrane vesicles. Biochim Biophys Acta 1104: 55-62, 1992.

17. Dudrick, P S, Bland K I, and Souba W W. Effects of tumor necrosis factor on system ASC-mediated glutamine transport by human fibroblasts. J Surg Res 52: 347-352, 1992.

18. Eagle, H. Nutritional needs of mammalian cells in tissue culture. Science 122: 501-504, 1955.

19. Fei, Y J, Sugawara M, Nakanishi T, Huang W, Wang H, Prasad P D, Leibach F H, and Ganapathy V. Primary structure, genomic organization, and functional and electrogenic characteristics of human system N 1, a Na⁺- and H⁺-coupled glutamine transporter. J Biol Chem 275: 23707-23717, 2000.

20. Fogh, J, and Trempe G. New human tumor cell lines. In: Human Tumor Cells in Vitro, edited by Fogh J. New York: Plenum, 1975, p. 115-159.

21. Gazzola, G C, DalrAsta V, Franchi-Gazzola R, and White M F. The cluster-tray method for rapid measurement of solute fluxes in adherent cultured cells. Anal Biochem 115: 368-374, 1981.

22. Gazzola, G C, Dall'Asta V, and Guidotti G G. The transport of neutral amino acids in cultured human fibroblasts. J Biol Chem 255: 929-936, 1980.

23. Hatanaka, T, Huang W, Ling R, Prasad P D, Sugawara M, Leibach F H, and Ganapathy V. Evidence for the transport of neutral as well as cationic amino acids by ATA3, a novel and liver-specific subtype of amino acid transport system A. Biochim Biophys Acta 1510: 10-17, 2001.

24. Hatanaka, T, Huang W, Martindale R G, and Ganapathy V. Differential influence of cAMP on the expression of the three subtypes (ATA1, ATA2, and ATA3) of the amino acid transport system A. FEBS Lett 505: 317-320, 2001.

25. Hatanaka, T, Huang W, Wang H, Sugawara M, Prasad P D, Leibach F H, and Ganapathy V. Primary structure, functional characteristics and tissue expression pattern of human ATA2, a subtype of amino acid transport system A. Biochim Biophys Acta 1467: 1-6, 2000.

26. Haussinger, D, Lamers W H, and Moorman A F. Hepatocyte heterogeneity in the metabolism of amino acids and ammonia. Enzyme (Basel) 46: 72-93, 1992.

27. Haussinger, D, Soboll S, Meijer A J, Gerok W, Tager J M, and Sies H. Role of plasma membrane transport in hepatic glutamine metabolism. Eur J Biochem 152: 597-603, 1985.

28. He, L, Isselbacher K J, Wands J R, Goodman H M, Shih C, and Quaroni A. Establishment and characterization of a new human hepatocellular carcinoma cell line. In Vitro 20: 493-504, 1984.

29. Hirayama, C, Suyama K, Horie Y, Tanimoto K, and Kato S. Plasma amino acid patterns in hepatocellular carcinoma. Biochem Med Metab Biol 38: 127-133, 1987.

30. Kekada, R, Prasad P D, Fei Y J, Torres-Zamorano V, Sinha S, Yang-Feng T L, Leibach F H, and Ganapathy V. Cloning of the sodium-dependent, broad-scope, neutral amino acid transporter B⁰ from a human placental choriocarcinoma cell line. J Biol Chem 271: 18657-18661, 1996.

31. Kekuda, R, Torres-Zamorano V, Fei Y J, Prasad P D, Li H W, Mader L D, Leibach F H, and Ganapathy V. Molecular and functional characterization of intestinal Na⁺-dependent neutral amino acid transporter B⁰. Am J Physiol Gastrointest Liver Physiol 272: G1463-G1472, 1997.

32. Kilberg, M S, Handlogten M E, and Christensen H N. Characteristics of an amino acid transport system in rat liver for glutamine, asparagine, histidine, and closely related analogs. J Biol Chem 255: 4011-4019, 1980.

33. Low, S Y, Salter M, Knowles R G, Pogson C I, and Rennie M J. A quantitative analysis of the control of glutamine catabolism in rat liver cells. Use of selective inhibitors. Biochem J 295: 617-624, 1993.

34. MacNab, G M, Alexander J J, Lecatsas G, Bey E M, and Urbanowicz J M. Hepatitis B surface antigen produced by a human hepatoma cell line. Br J Cancer 34: 509-515, 1976.

35. Mailliard, M E, and Kilberg M S. Sodium-dependent neutral amino acid transport by human liver plasma membrane vesicles. J Biol Chem 265: 14321-14326, 1990.

36. Matsuno, T. Pathway of glutamate oxidation and its regulation in the HuH13 line of human hepatoma cells. J Cell Physiol 148: 290-294, 1991.

37. Matsuno, T, and Goto I. Glutaminase and glutamine synthetase activities in human cirrhotic liver and hepatocellular carcinoma. Cancer Res 52: 1192-1194, 1992.

38. Matsuno, T, and Hirai H. Glutamine synthetase and glutaminase activities in various hepatoma cells. Biochem Int 19: 219-225, 1989.

39. Medina, M A, Sanchez-Jimenez F, Marquez J, Rodriguez Quesada A, and Nunez de Castro I. Relevance of glutamine metabolism to tumor cell growth. Mol Cell Biochem 113: 1-15, 1992.

40. Meijer, A J, Lamers W H, and Chamuleau RAFM Nitrogen metabolism and ornithine cycle function. Physiol Rev 70: 701-748, 1990.

41. Nakabayashi, H, Taketa K, Yamane T, Miyazaki M, Miyano K, and Sato J. Phenotypical stability of a human hepatoma cell line, Huh-7, in long-term culture with chemically defined medium. GANN 75: 151-158, 1984.

42. Nakanishi, T, Sugawara M, Huang W, Martindale R G, Leibach F H, Ganapathy M E, Prasad P D, and Ganapathy V. Structure, function, and tissue expression pattern of human SN2, a subtype of the amino acid transport system N. Biochem Biophys Res Commun 281: 1343-1348, 2001.

43. Pawlik, T M, Rubin A I, Souba W W, and Bode B P. Liver tumor cell nutrient uptake as a function of the cell cycle. Surg. Forum L: 23-25, 1999.

44. Pawlik, T M, Souba W W, Sweeney T J, and Bode B P. Amino acid uptake and regulation in multicellular hepatoma spheroids. J Surg Res 91: 15-25, 2000.

45. Pfeifer, A M, Cole K E, Smoot D T, Weston A, Groopman J D, Shields P G, Vignaud J M, Juillerat M, Lipsky M M, Trump B F, Lechner J F, and Harris C C. Simian virus 40 large tumor antigen-immortalized normal human liver epithelial cells express hepatocyte characteristics and metabolize chemical carcinogens. Proc Natl Acad Sci USA 90: 5123-5127, 1993.

46. Scharf, J G, Dombrowski F, and Ramadori G. The IGF axis and hepatocarcinogenesis. Mol Pathol 54: 138-144, 2001.

47. Sebolt, J S, and Weber G. Negative correlation of L-glutamine concentration with proliferation rate in rat hepatomas. Life Sci 34: 301-306, 1984.

48. Shafqat, S, Tamarappoo B K, Kilberg M S, Puranam R S, McNamara J O, Guadano-Ferraz A, and Fremeau R T, Jr. Cloning and expression of a novel Na⁺-dependent neutral amino acid transporter structurally related to mammalian Na⁺/glutamate cotransporters. J Biol Chem 268: 15351-15355, 1993.

49. Stevens, B. Amino acid transport in intestine. In: Mammalian Amino Acid Transport, edited by Kilberg M, and Haussinger D. New York: Plenum, 1992, p. 149-163.

50. Torres-Zamorano, V, Leibach F H, and Ganapathy V. Sodium-dependent homo- and hetero-exchange of neutral amino acids mediated by the amino acid transporter ATB⁰. Biochem Biophys Res Commun 245: 824-829, 1998.

51. Utsunomiya-Tate, N, Endou H, and Kanai Y. Cloning and functional characterization of a system ASC-like Na⁺-dependent neutral amino acid transporter. J Biol Chem 271: 14883-14890, 1996.

52. Wasa, M, Bode B P, Abcouwer S F, Collins C L, Tanabe K K, and Souba W W. Glutamine as a regulator of DNA and protein biosynthesis in human solid tumor cell lines. Ann Surg 224: 189-197, 1996.

53. Labow, B. I. and W. W. Souba (2000). “Glutamine.” World Journal of Surgery 24(12): 1503-13.

54. Darmaun, D., D. E. Matthews, et al. (1986). “Glutamine and glutamate kinetics in humans.” Am J Physiol 251(1 Pt 1): E117-26.

55. Haussinger, D. (1990). “Nitrogen metabolism in liver: structural and functional organization and physiological relevance.” Biochemical Journal 267(2): 281-90.

56. Abcouwer, S. F. “Effects of glutamine on immune cells.” Nutrition 16: 67-69, 2000.

57. Bode, B. P., B. C. Fuchs, et al. (2002). “Molecular and functional analysis of glutamine uptake in human hepatoma and liver-derived cells.” American Journal of Physiology. Gastrointestinal and Liver Physiology 283(5): G1062-73.

58. Tailor, C. S., A. Nouri, et al. (1999). “A sodium-dependent neutral-amino-acid transporter mediates infections of feline and baboon endogenous retroviruses and simian type D retroviruses.” Journal of Virology 73(5): 4470-4.

59. Ogunbiyi, J. O. (2001). “Hepatocellular carcinoma in the developing world.” Seminars in Oncology 28(2): 179-87.

60. Kang, M. A., K. Y. Kim, et al. (2000). “The growth inhibition of hepatoma by gene transfer of antisense vascular endothelial growth factor.” Journal of Gene Medicine 2(4): 289-96.

61. Wasa, M., B. P. Bode, et al. (1996). “Adaptive regulation of amino acid transport in nutrient-deprived human hepatomas.” American Journal of Surgery 171(1): 163-9.

62. Clemens, M. J. (1997). “PKR—a protein kinase regulated by double-stranded RNA.” The International Journal of Biochemistry & Cell Biology 29(7): 945-9.

63. Rothstein, J. D., M. Dykes_Hoberg, et al. (1996). “Knockout of glutamate transporters reveals a major role for astroglial transport in excitotoxicity and clearance of glutamate.” Neuron 16(3): 675-86.

64. Simantov, R., W. Liu, et al. (1999). “Antisense knockdown of glutamate transporters alters the subfield selectivity of kainate-induced cell death in rat hippocampal slice cultures.” Journal of Neurochemistry 73(5): 1828-35.

65. Mates, J. M., C. Perez_Gomez, et al. (2002). “Glutamine and its relationship with intracellular redox status, oxidative stress and cell proliferation/death.” The International Journal of Biochemistry & Cell Biology 34(5): 439-58.

66. Roth, E., R. Oehler, et al. (2002). “Regulative potential of glutamine—relation to glutathione metabolism.” Nutrition (Burbank, Los Angeles County, Calif.) 18(3): 217-21.

67. Williams, B. R. (1999). “PKR; a sentinel kinase for cellular stress.” Oncogene 18(45): 6112-20.

68. Shir, A. and A. Levitzki (2002). “Inhibition of glioma growth by tumor-specific activation of double-stranded RNA#150;dependent protein kinase PKR.” Nature Biotechnology 20(9): 895-900.

69. Wek, R. C. (1994). “eIF-2 kinases: regulators of general and gene-specific translation initiation.” Trends in Biochemical Sciences 19(11): 491-6.

70. de Haro, C., R. Mendez, et al. (1996). “The eIF-2alpha kinases and the control of protein synthesis.” The Faseb Journal: Official Publication of the Federation of American Societies For Experimental Biology 10(12): 1378-87.

71. Hinnebusch, A. G. (1994). “The eIF-2 alpha kinases: regulators of protein synthesis in starvation and stress.” Seminars in Cell Biology 5(6): 417-26.

72. Berlanga, J. J., J. Santoyo, et al. (1999). “Characterization of a mammalian homolog of the GCN2 eukaryotic initiation factor 2alpha kinase.” European Journal of Biochemistry 265(2): 754-62.

73. Sood, R., A. C. Porter, et al. (2000). “A mammalian homologue of GCN2 protein kinase important for translational control by phosphorylation of eukaryotic initiation factor-2alpha.” Genetics 154(2): 787-801.

What is claimed is:

1. A method of inducing apoptosis of a cell, comprising contacting a cell with an agent, wherein (a) the agent inhibits the uptake of glutamine by the cell, and (b) the cell undergoes apoptosis.

2. The method of claim 1, wherein the cell is a carcinoma cell.

3. The method of claim 2, wherein the cell is a hepatocarcinoma cell.

4. The method of claim 2 wherein the carcinoma cell is in a patient.

5. The method of claim 3, wherein the cell is selected from the group consisting of PLC/PRF/5, SK-Hep, Hep3B, Huh-7, FOCUS and HepG2 cell lines.

6. The method of claim 1, wherein said agent modulates a component of a glutamine transport system.

7. The method of claim 6, wherein the component of a glutamine transport system is ATB⁰.

8. The method of any one of claims 1 wherein the agent inhibits ATB⁰ activity.

9. The method of claim 8 wherein the agent is selected from the group consisting of an antibody, a polynucleotide, and an amino acid analog.

10. The method of claim 8 wherein the agent is a polynucleotide that inhibits the expression of ATB⁰.

11. The method of claim 10 wherein the polynucleotide comprises a sequence set forth in SEQ ID NO:2, SEQ ID NO:3, SEQ ID NO:4, SEQ ID NO:5, or SEQ ID NO:6.

12. The method of claim 10 wherein the polynucleotide consists essentially of a sequence set forth in SEQ ID NO:2, SEQ ED NO:3, SEQ ID NO:4, SEQ ID NO:5, or SEQ D NO:6.

13. The method of claim 11 wherein the polynucleotide comprises a sequence as set forth in SEQ ID NO:3.

14. The method of claim 12 wherein the polynucleotide consists essentially of a sequence as set forth in SEQ ID NO:3.

15. A method of inducing apoptosis of a cell, comprising contacting a cell with a vector which comprises a polynucleotide that encodes a polynucleotide which reduces the expression of an ATB⁰ gene product, wherein (a) the vector enters the cell, (b) the polynucleotide is produced in the cell

16. The method of claim 15 wherein the polynucleotide comprises a sequence of at least 10 contiguous nucleotides from SEQ ID NO:1.

17. The method of claim 16 wherein the polynucleotide comprises a sequence as set forth in any one of SEQ ID NO:2, SEQ ID NO:3, SEQ ID NO:4, SEQ ID NO:5 and SEQ ID NO:6.

18. The method of claim 16 wherein the polynucleotide consists essentially of a sequence as set forth in any one of SEQ ID NO:2, SEQ ID NO:3, SEQ ID NO:4, SEQ ID NO:5 and SEQ ID NO:6.

19. The method of claim 15 wherein the polynucleotide consists essentially of SEQ ID NO:3.

20. The method of any one of claims 19 wherein the cell is a hepatocarcinoma cell.

21. The method of claim 20 wherein the hepatocarcinoma cell is in a patient.

22. The method of claim 15 wherein the vector is an adenovirus vector.

23. The method of claim 15 wherein the vector is an adenovirus vector.

24. A method of treating an hepatocarcinoma comprising administering a therapeutically effective amount of an agent to an individual, wherein (a) the agent contacts a hepatoma cell in the individual, (b) the agent selectively inhibits the activity of an ATB⁰ of the hepatoma cell, (c) glutamine uptake by the hepatoma cell is significantly reduced, and (d) the hepatoma cell undergoes apoptosis.

25. The method of claim 24 wherein the agent comprises (a) a polynucleotide having a sequence set forth in any one of SEQ ID NO:2, SEQ ID NO:3, SEQ ID NO:4, SEQ ID NO:5, or SEQ ID NO:6, wherein the polynucleotide is operably linked to a promoter in an adenovirus vector.

26. A method of diagnosing cancer in a patient comprising obtaining a sample from the patient, determining the amount of ATB⁰ in the sample, and predicting whether a carcinoma is in the patient based upon a higher than normal level of ATB⁰ in the sample.

27. The method of claim 26 wherein the carcinoma is a hepatoma.
